SEQUENCE LISTING (18 sequences)

SEQ ID NO: 1 - DNA, 19 bp, Artificial
  PCR primer
gtcaaggaagctaagactg

SEQ ID NO: 2 - DNA, 20 bp, Artificial
  PCR primer
tgttaggatctgggccaaag

SEQ ID NO: 3 - DNA, 20 bp, Artificial
  PCR primer
tgatcctggaaggggaagac

SEQ ID NO: 4 - DNA, 22 bp, Artificial
  PCR primer
cacgaggcagatactttcaaac

SEQ ID NO: 5 - DNA, 20 bp, Artificial
  PCR primer
aagtgcgagccaaggaccag

SEQ ID NO: 6 - DNA, 24 bp, Artificial
  PCR primer
ttacgagcagtgatgcctaccaac

SEQ ID NO: 7 - DNA, 20 bp, Artificial
  PCR primer
ccttcaccatggacgacatg

SEQ ID NO: 8 - DNA, 19 bp, Artificial
  PCR primer
cggctttggtcacgtcact

SEQ ID NO: 9 - DNA, 22 bp, Artificial
  PCR primer
agtactgccgactgcagaagtg

SEQ ID NO: 10 - DNA, 22 bp, Artificial
  PCR primer
tgtttcggtcgtttctcacaga

SEQ ID NO: 11 - DNA, 24 bp, Artificial
  PCR primer
caaatcatcagggtaccactatgg

SEQ ID NO: 12 - DNA, 23 bp, Artificial
  PCR primer
ctgaatacttctgcggaaaaagc

SEQ ID NO: 13 - DNA, 20 bp, Artificial
  PCR primer
tgccggctacagaagtgctt

SEQ ID NO: 14 - DNA, 21 bp, Artificial
  PCR primer
cttcttgttccggtcatttcg

SEQ ID NO: 15 - DNA, 18 bp, Artificial
  PCR primer
agcagcagggacggtttg

SEQ ID NO: 16 - DNA, 25 bp, Artificial
  PCR primer
gatgctctagacacttaaggccaat

SEQ ID NO: 17 - DNA, 18 bp, Artificial
  PCR primer
tttcccgcaggctatgga

SEQ ID NO: 18 - DNA, 19 bp, Artificial
  PCR primer
tgctgatgggctcatggat

(19) United States
(12) Patent Application Publication
(10) Pub. No.: US 2006/0074109 A1
(43) Pub. Date: Apr. 6, 2006

(54) Use of inhibitors of 24-hydroxylase in the treatment of cancer

(75) Inventors: William J. Polvino, Trinton Falls, NJ (US); Timo Ylikomi, Tampere (FI); Yan-Ru Lou, Tampere (FI); Merja Ahonen, Jarvimaa (FI); Pentti Tuohimaa, Tampere (FI); Susanna Miettinen, Siivikkala (FI)

Correspondence Address: PALMER & DODGE, LLP;KATHLEEN M. WILLIAMS, 111 HUNTINGTON AVENUE, BOSTON, MA, 02199, US

(73) Assignee: Sapphire Therapeutics, Inc.

(21) Appl. No.: 11/234,941

(22) Filed: Sep. 26, 2005

Related U.S. Application Data
(60) Provisional application No. 60/612,714, filed on Sep. 24, 2004.

(30) Foreign Application Priority Data
Oct. 19, 2004 (WO) PCT/EP04/11951

(51) Int. Cl. A61K 31/4439; A61K 31/4178; A61K 31/4172

(52) U.S. Cl. 514/341; 514/400; 514/397

(57) ABSTRACT

The present invention relates to a method of treating cancer in a subject. The method comprises administering to a subject suffering from cancer a therapeutically effective amount of a 24-hydroxylase inhibitor. In certain embodiments, the 24-hydroxylase inhibitor can be coadministered with calcitriol.

### RELATED APPLICATIONS

This application claims the benefit of U.S. Provisional Application No. 60/612,714, filed on Sep. 24, 2004 and PCT/EP04/011951, filed Oct. 19, 2004. The entire teachings of that application are incorporated herein by reference.

### BACKGROUND OF THE INVENTION

Cancer is a disease for which many potentially effective treatments are available. However, due to the prevalence of cancers of various types and the serious disease effects, more effective treatments, for example, those with fewer adverse side effects or more successful treatment outcomes, are needed.

Vitamin D is known to play multiple roles. It is best known for its ability to raise the level of plasma calcium by stimulating bone resorption and intestinal calcium absorption. Vitamin D also has been suggested to play a role in the immune system and the reproductive system. Vitamin D has been shown to down regulate the renin-angiotensin system that in turn regulates blood pressure. In addition, vitamin D and its analogs have been shown to inhibit the proliferation of certain cells, for example, certain types of cancer cells.

### SUMMARY OF THE INVENTION

The present invention relates to a method of treating cancer in a subject. The method comprises administering to a subject suffering from cancer a therapeutically effective amount of a 24-hydroxylase inhibitor.

The inhibitor can be a compound selected from the group consisting of azoles, aminoalkanimidazoles, aminoalkantriazoles, acylated aminoalkanimidazoles, and acylated aminoalkantriazoles. The inhibitor can be an azole compound having a bulky substituent attached at the C-alpha position to the azole. In some embodiments the inhibitor at the C-alpha position is phenyl, naphthyl, thienyl, or pyridyl. The phenyl, naphthyl, thienyl or pyridyl group can be monosubstituted by halogen, (C₁₋₄)alkoxy, (C₁₋₄)alkyl, di-(C₁₋₄)alkylamino or cyano.

In some embodiments the inhibitor is selected from (R)-SDZ-286907, (R)-SDZ-287871, (R)-VAB636, (R)-VID400, and (S)-SDZ-285428. These compounds are depicted in FIG. 10 as compounds Ia, Ib, Ic, Id, and Ie, respectively.

In a particular embodiment, the 24-hydroxylase inhibitor is represented by the structural Formula I: 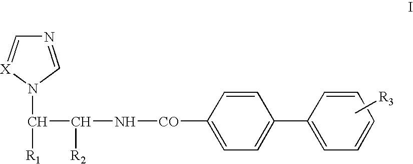
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₁₄)alkyl, di-(C₁₋₄)alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

The acylated aminoalkanimidazoles and aminoalkantriazoles of Formula I are fully described in U.S. Pat. No. 5,622,982 to Schuster et al., the entire content of which is hereby incorporated by reference.

In another embodiment, the 24-hydroxylase inhibitor is represented by the structural Formula II: 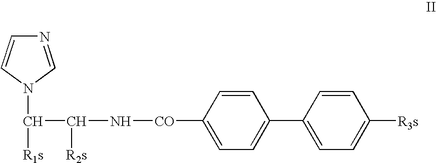
or a pharmaceutically acceptable salt, solvate or hydrate thereof, wherein:
R₁ₛ is phenyl, phenyl monosubstituted by halogen, or 1-naphtyl, and R₂ₛ is hydrogen; or
R₁ₛ is hydrogen and R₂ₛ is pyridyl or 2-(5-chloro)pyridyl; and
R₃ₛ is halogen, (C₁₋₄) alkoxy.

The compounds of Formula II are fully described in U.S. Pat. No. 5,622,982 to Schuster et al.

In certain embodiments the inhibitor is a structural analog of 1,25-(OH)₂ vitamin D₃. For example, the inhibitor may be represented by Formula IV: 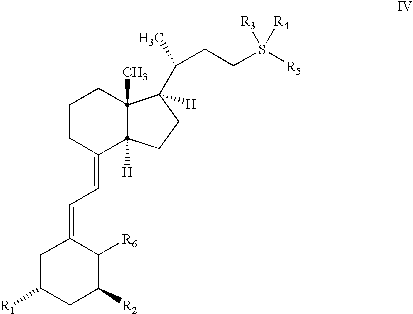
wherein R₁ and R₂ are each independently selected from the group consisting of hydrogen, OR′, —C(O)H, and —C(O)R′;
wherein R′ is selected from the group consisting of a C1 to C₆ alkyl, a cycloalkyl, phenyl, an alkylaryl, an arylalkyl, and a heteroaryl; each of which can be optionally substituted with at least one halogen, thiol, mercapto, hydroxyl, or amino group;
wherein R₃, R₄ and R₅ are each independently selected from the group consisting of hydrogen, hydroxyl, oxy, imine, phenyl, a C1 to C₆ alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R₆ is hydrogen, ═CH₂ or a C₁ to C₆ alkyl, alkenyl, cycloalkyl, or cycloalkenyl, each of which can be optionally substituted with at least one halogen, thiol, mercapto, hydroxyl, or amino group; or a pharmaceutically acceptable salt, hydrate, solvate, ester, or isomer thereof.

Or, the inhibitor may be represented by Formula V: 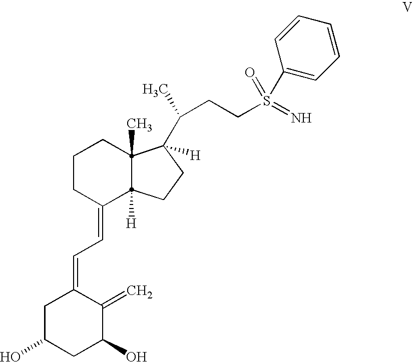
or a pharmaceutically acceptable salt, solvate, hydrate, ester or isomer thereof.

Or, the inhibitor may be represented by Formula VI: 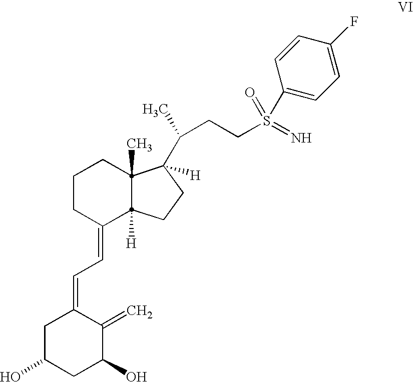
or a pharmaceutically acceptable salt, solvate, hydrate, ester or isomer thereof.

In a particular embodiment, the 24-hydroxylase inhibitor is coadministered with calcitriol.

The invention further relates to pharmaceutical composition useful for the treatment of cancer comprising a 24-hydroxylase inhibitor. In a particular embodiment, the pharmaceutical composition further comprises calcitriol. The 24-hydroxylase inhibitor and the calcitriol can each be present in the pharmaceutical composition in a therapeutically effective amount. In another aspect, the 24-hydroxylase inhibitor and the calcitriol together comprise a therapeutically effective amount. The pharmaceutical composition of the present invention can optionally contain a pharmaceutically acceptable carrier.

The invention further relates to use of a 24-hydroxylase inhibitor for the manufacture of a medicament for treating cancer.

### BRIEF DESCRIPTION OF THE DRAWINGS

FIGS. 1A, 1B and 1C are graphs showing the effect of 25(OH)D₃, 1,25-(OH)₂D₃ and EB 1098, respectively, on the growth of OVCAR-3 cells following treatment at the indicated concentrations for 11 days.

FIG. 2 is a scan of a gel electrophoresis showing the expression of 1αOHase in OVCAR-3 cells. A RT-PCR was used for the detection of 1αOHase mRNA from OVCAR-3 cells. A 303 bp band can be seen in the 1αOHase-transfected COS sample (lane 3) and in both ethanol-treated (lanes 7 and 8) and 100 nM 1,25-(OH)2D3-treated (lanes 9 and 10) OVCAR-3 samples. In lane 4, there is a negative control for the ethanol-treated sample, and lane 5 represents a negative control for the 1,25-(OH)2D3-treated sample. Lane 1 is a 100 bp marker, lane 2 is a RT-PCR functional control (1100 bp), and lane 6 is empty.

FIG. 3 is a graph showing the relative expression ratios of 24OHase mRNA in OVCAR-3 cells after 6 or 24 hr treatment with 100 nM 1,25-(OH)2D3, 25(OH)D3, EB 1098 or ethanol (vehicle). A quantitative RT-PCR was done using 0.3 μg total RNA. The human keratinocyte cell line, HaCaT, was used as an expression control of 24OHase. The values represent the mean of 2 independent experiments ±SD.

FIG. 4A is a graph showing the effect of the 24OHase inhibitor, VID 400, on the cell-growth response to 25(OH)D3.

FIG. 4B is a graph showing the effect of the 24OHase inhibitor, VID 400, on the cell-growth response to 1,25-(OH)2D3.

FIG. 5A is a scan of a gel electrophoresis showing one RPA experiment (cell line UT-OC-2 not shown). Receptors, cell lines and different treatments (C=vehicle, D₃=100 nM 1,25(OH)₂D₃, EB=100 nM EB 1089, A=10 μM ATRA and 9C=10 μM 9-CRA) are indicated. The label N is a negative control (yeast total RNA) and E is a probe excess control (32 μg sample RNA).

FIG. 5B is a bar graph showing quantified expressions of the VDR receptor (combination of RPA and RT-PCR data, the mean of three experiments ±SD). The basal expression level of individual receptor in UT-OC-4 was set at 100 and expressions in other cell lines compared to this. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 5C is a bar graph showing quantified expressions of the RARα receptor (combination of RPA and RT-PCR data, the mean of three experiments ±SD). The basal expression level of individual receptor in UT-OC-4 was set at 100 and expressions in other cell lines compared to this. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 5D is a bar graph showing quantified expressions of the RARβ receptor (combination of RPA and RT-PCR data, the mean of three experiments ±SD). The basal expression level of individual receptor in UT-OC-4 was set at 100 and expressions in other cell lines compared to this. The Y-axis continues from 150 after the break. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 5E is a bar graph showing quantified expressions of the RARγ receptor (combination of RPA and RT-PCR data, the mean of three experiments ±SD). The basal expression level of individual receptor in UT-OC-4 was set at 100 and expressions in other cell lines compared to this. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 5F is a bar graph showing quantified expressions of the RXRα receptor (combination of RPA and RT-PCR data, the mean of three experiments ±SD). The basal expression level of individual receptor in UT-OC-4 was set at 100 and expressions in other cell lines compared to this. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 5G is a bar graph showing quantified expressions of the RXRβ receptor (combination of RPA and RT-PCR data, the mean of three experiments ±SD). The basal expression level of individual receptor in UT-OC-4 was set at 100 and expressions in other cell lines compared to this. The arrows show the up (↑) or down (↓) regulation of receptor expression in the cell lines and hormone treatments (24 hour, 100 nM 1,25(OH)₂D₃, 100 nM EB 1089, 10 μM ATRA or 10 μM 9-CRA).

FIG. 6A is a scan of a gel electrophoresis showing the basal expression levels of the indicated nuclear receptor cofactors in ovarian cancer cell lines which were determined from 8 μg total RNA samples using RPA. The negative control (yeast total RNA, lane 1) and cell lines UT-OC-1 (lane 2), UT-OC-2 (lane 3), UT-OC-3 (lane 4), UT-OC-4 (lane 5), UT-OC-5 (lane 6), SK-OV-3 (lane 7), OVCAR-3 (lane 8) and MCF-7 (lane 10) are indicated. Line 9 represents probe excess control (32 μg RNA). In UT-OC-4 cells the basal expression of an individual cofactor (B-H) was set at 100 and the expressions in other cell lines compared to this. The values represent the mean of three separate experiments ±SD.

FIG. 6B is a bar graph showing quantified basal expression levels of NCoR mRNA.

FIG. 6C is a bar graph showing quantified basal expression levels of SMRT.

FIG. 6D is a bar graph showing quantified basal expression levels of TIF2.

FIG. 6E is a bar graph showing quantified basal expression levels of AIB1. An arrow (↑) indicates the up-regulation of AIB1 expression by ATRA and 9-CRA in OVCAR-3 cells.

FIG. 6F is a bar graph showing basal expressions of pCAF.

FIG. 6G is a bar graph showing basal expressions of CBP.

FIG. 6H is a bar graph showing basal expressions of p300.

FIG. 7A is bar graph showing the relative expression of 24OHase mRNA in ovarian cancer cells after 24 h treatment with (A) 100 nM 1,25(OH)₂D₃ and 100 nM EB 1098 or (B) 10 μM ATRA and 10 μM 9-CRA. The basal expression level of 24OHase in UT-OC-4 was set at 100 and the expressions in other cell lines and treatments compared to this sample. The values represent a combination of RPA and RT-PCR data (the mean of three experiments ±SD). In FIG. 7A the Y-axis continues from 10 after the first break and from 750 after the second break.

FIG. 7B is bar graph showing the relative expression of 24OHase mRNA in ovarian cancer cells after 24 h treatment with (A) 100 nM 1,25(OH)₂D₃ and 100 nM EB 1098 or (B) 10 μM ATRA and 10 μM 9-CRA. The basal expression level of 24OHase in UT-OC-4 was set at 100 and the expressions in other cell lines and treatments compared to this sample. The values represent a combination of RPA and RT-PCR data (the mean of three experiments ±SD). In FIG. 7B the Y-axis continues from 125 after the break.

FIG. 8A is a bar graph showing the effect of 24OHase inhibitor on the cell growth response to 1,25(OH)₂D₃, EB 1089, ATRA and 9-CRA in UT-OC-1 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone ±VID 400-treated sample is indicated by *. The lines and * indicate statistically significant differences between VID 400 alone and hormone+VID 400-treated samples (P<0.05, Student's t-test).

FIG. 8B is a bar graph showing the effect of 24OHase inhibitor on the cell growth response to 1,25(OH)₂D₃, EB 1089, ATRA and 9-CRA in UT-OC-2 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIG. 8C is a bar graph showing the effect of 24OHase inhibitor on the cell growth response to 1,25(OH)₂D₃, EB 1089, ATRA and 9-CRA in UT-OC-3 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIG. 8D is a bar graph showing the effect of 24OHase inhibitor on the cell growth response to 1,25(OH)₂D₃, EB 1089, ATRA and 9-CRA in UT-OC-4 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIG. 8E is a bar graph showing the effect of 24OHase inhibitor on the cell growth response to 1,25(OH)₂D₃, EB 1089, ATRA and 9-CRA in UT-OC-5 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIG. 8F is a bar graph showing the effect of 24OHase inhibitor on the cell growth response to 1,25(OH)₂D₃, EB 1089, ATRA and 9-CRA in SK-OV-3 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIG. 8G is a bar graph showing the effect of 24OHase inhibitor on the cell growth response to 1,25(OH)₂D₃, EB 1089, ATRA and 9-CRA in OVCAR-3 cell lines. Cells were treated with indicated hormone concentrations or combinations of hormone and 24OHase inhibitor (VID 400). The growth medium and hormones were changed every third day. After the 11 days treatment the cells were fixed and stained with crystal violet and the optical density (590 nm) determined. The cell growth is presented as a percentage of ethanol-treated cells (100%). The values represent the mean of three separate experiments ±SD. Statistically significant difference between hormone alone and hormone+VID 400-treated sample is indicated by *. The lines and * indicate statistically significant differences between VID 400 alone and hormone+VID 400-treated samples (P<0.05, Student's t-test).

FIGS. 9A-9D show the results of 24OHase inhibitor-1,25(OH)₂D₃ coadministration on cell growth in four different cancer cell lines. OVCAR-3 ovarian cancer cells are shown in FIG. 9A, and three prostate cancer cell lines (CWR22Rv-1, PC3, and DU145) are shown in FIGS. 9B-9D. Details of the experiment are described under Experiment VI below.

FIGS. 10A and 10B depict the structures of selected azole-type 24-hydroxylase inhibitors.

FIGS. 11A and 11B depict the structures of selected analogs of 1,25-(OH)₂ vitamin D₃ which are useful as 24-hydroxylase inhibitors.

### DETAILED DESCRIPTION OF THE INVENTION

The present invention relates to a method of treating cancer in a subject. The method comprises administering to a subject suffering from cancer a therapeutically effective amount of a 24-hydroxylase inhibitor.

In a particular embodiment, the 24-hydroxylase inhibitor is coadministered with calcitriol.

As defined herein, cancer refers to tumors, neoplasms, carcinomas, sarcomas, leukemias, lymphomas and the like. Suitable cancers include, but are not limited to, colorectal cancer, esophageal cancer, myelodysplastic syndromes, multiple myeloma, gliomas, non-small cell lung cancer, stomach cancer, acute myeloid leukemia, hepatocellular carcinoma, breast cancer, ovarian cancer and prostate cancer.

Colorectal Cancer

Cancer of the large intestine and rectum (colorectal cancer) is the second most common type of cancer and the second leading cause of cancer death in Western countries. It develops as the result of a pathologic transformation of normal colon epithelium to an invasive cancer.

Esophageal Cancer

The esophagus is a muscular tube that connects the mouth to the stomach and carries food to the stomach. There are two main types of esophageal cancer: squamous cell carcinoma and adenocarcinoma. At one time, squamous cell carcinoma was by far the more common of the two cancers and was responsible for almost 90% of all esophageal cancers. However, more recent medical studies show that squamous cell cancers make up less than 50% of esophageal cancers today. Still, squamous cell carcinoma remains one of the most common neoplasms in the world, affecting approximately 350,000 people annually worldwide (Parkin et al,. (1993) Int. J. Cancer 54: 594-606). Tobacco and alcohol are two major etiological factors in oral cavity squamous cell carcinoma (Binnie et al. (1983) J. Oral Pathol., 12: 11-29).

Myelodysplastic Syndromes

Myelodysplastic syndromes (MDS) are a heterogeneous group of conditions caused by abnormal blood-forming cells of the bone marrow. In MDS the bone marrow cannot produce blood cells effectively, and many of the blood cells formed are defective. These abnormal blood cells are usually destroyed before they leave the bone marrow or shortly after entering the bloodstream. As a result, patients have shortages of blood cells, which are reflected in their low blood counts. About twenty percent of cases arise in patients who have received either chemotherapy or radiotherapy as part of their treatment for another disease.

Although MDS has not been considered cancer in the past, most hematologists (specialists in diseases of the blood) now consider it is a form of cancer. The major reason is that it is considered a clonal disease with a single population of abnormal cells. That means that all the cells are exactly alike. This is often seen in cancer where all the cells have started from an original abnormal cell. A second reason is that in about 30% of MDS cases, the abnormal bone marrow cells eventually progress into acute leukemia, a rapidly growing cancer of bone marrow cells. Some doctors think MDS is an early form of leukemia although it may never progress into leukemia.

Multiple Myeloma

Multiple myeloma is a type of cancer formed by cancerous plasma cells in the blood. Normal plasma cells are an important part of the body's immune system.

When plasma cells grow out of control, they can form a tumor called myeloma. Myeloma tumors can grow in many places, including bone marrow. Tumors that grow in more than one place are called multiple myeloma. The myeloma cells interfere with the functions of the bone marrow to make red blood cells, platelets, and white blood cells. According to the International Myeloma Foundation, there are over 13,500 new cases of myeloma in the U.S. each year, representing twenty percent of blood cancers, and one percent of all types of cancer.

Gliomas

Gliomas are primary brain tumors which arise from the glial cells in the brain and spinal cord, and are the most common primary brain tumors. Gliomas are classified into several groups based on the type of glial cell involved. For example, astrocytomas, which are the most common type of gliomas, are developed from astrocytes. Types of astrocytomas include well-differentiated, anaplastic, and glioblastoma multiforme. Other types of glioma include ependymomas, oligodendrogliomas, ganglioneuromas, mixed gliomas, brain stem gliomas, optic nerve gliomas, meningiomas, pineal tumors, pituitary adenomas, and primitive neuroectodermal tumors, such as medulloblastomas, neuroblastomas, pineoblastomas, medulloepitheliomas, ependymoblastomas and polar spongioblastomas.

Non-Small Cell Lung Cancer

Non-small cell lung cancer (NSCLC) is the most common type of lung cancer, and is a heterogeneous aggregate of at least 3 distinct histologies of lung cancer including epidermoid or squamous carcinoma, adenocarcinoma, and large cell carcinoma.

Stomach Cancer

Stomach cancer is the second most common human malignancy in the world. About 99% of stomach cancers are adenocarcinomas. Other stomach cancers are leiomyosarcomas (cancers of the smooth muscle) and lymphomas. While the exact causes are not yet understood, a number of causes and risk factors have been associated with an increased risk of stomach cancer, including: Helicobacter pylori (H. pylori) infection, pernicious anaemia, a diet high in salt and foods that are smoked or cured, family history, type A blood group, smoking, and atrophic gastritis.

Acute Myeloid Leukemia

Acute myeloid (myelocytic, myelogenous, myeloblastic, myelomonocytic) leukemia is a life-threatening disease in which myelocytes (the cells that normally develop into granulocytes) become cancerous and rapidly replace normal cells in the bone marrow. The leukemic cells accumulate in the bone marrow and destroy and replace cells that form normal blood cells. They are released into the bloodstream and transported to the other organs where they continue to grow and divide.

Hepatocellular Carcinoma

Hepatocellular carcinoma (HCC) is a cancer that begins in the liver cells. HCCs are the most common type of cancer originating in the liver (primary liver cancer), and is one of the leading malignancies worldwide, especially prevalent in the Asia and Pacific regions. More than 1 million people develop into HCC each year (Bosch & Munoz. Epidemiology of hepatocellular carcinoma. In Bannsch & Keppler, eds. Liver cell carcinoma. Dordrecht: Kluwer Academic, 1989; 3-12). The five year survival rate of HCC is quite low (less than 5%). A number of etiological factors, particularly hepatitis B virus (HBV) infection, are involved in the occurrence and progression of HCC.

Breast Cancer

Breast cancer is classified by the kind of tissue in which it starts and by the extent of its spread. Breast cancer may start in the milk glands, milk ducts, fatty tissue, or connective tissue. Different types of breast cancers progress differently. Generalizations about particular types are based on similarities in how they are discovered, how they progress, and how they are treated. Some grow very slowly and spread to other parts of the body (metastasize) only after they become very large. Others are more aggressive, growing and spreading quickly. However, the same type of cancer may progress differently in different women.

In situ carcinoma, which means cancer in place, is an early cancer that has not invaded or spread beyond its point of origin. In situ carcinoma accounts for more than 15 percent of all breast cancers diagnosed in the United States.

About 90 percent of all breast cancers start in the milk ducts or milk glands. Ductal carcinoma in situ starts in the walls of milk ducts. It can develop before or after menopause. This type of cancer occasionally can be felt as a lump and may appear as tiny specks of calcium deposits (microcalcifications) on mammograms. Ductal carcinoma in situ is often detected by mammography before it is large enough to be felt. It is usually confined to a specific area of the and can be totally removed by surgery. If only the ductal carcinoma in situ is removed, about 25 to 35 percent of women develop invasive cancer, usually in the same breast.

Lobular carcinoma in situ, which starts in the milk glands, usually develops before menopause. This type of breast cancer, which cannot be felt or seen on mammograms, is usually found incidentally on mammography during investigation of a lump or other abnormality that is not lobular carcinoma in situ. Between 25 and 35 percent of women who have it develop invasive breast cancer eventually—sometimes after as long as 40 years—in the same or opposite breast or in both breasts.

Invasive breast cancers, which can spread to and destroy other tissues, may be localized (confined to the breast) or metastatic (spread to other parts of the body). About 80 percent of invasive breast cancers are ductal and about 10 percent are lobular. The prognosis for ductal and lobular invasive cancers is similar. Other less common types of cancer, such as medullary carcinoma and tubular carcinoma (which start in milk glands), have a somewhat better prognosis.

Ovarian Cancer

Ovarian cancer is cancer that begins in the cells that constitute the ovaries, including surface epithelial cells, germ cells, and the sex cord-stromal cells. Almost 70 percent of women with the common epithelial ovarian cancer are not diagnosed until the disease is advanced in stage—i.e., has spread to the upper abdomen (stage III) or beyond (stage 1V). The 5-year survival rate for these women is only 15 to 20 percent, whereas the 5-year survival rate for stage I disease patients approaches 90 percent and for stage II disease patients approaches 70 percent.

There are many types of tumors that can start in the ovaries. Some are benign, or noncancerous, and the patient can be cured by surgically removing one ovary or the part of the ovary containing the tumor. Some are malignant or cancerous. The treatment options and the outcome for the patient depend on the type of ovarian cancer and how far it has spread before it is diagnosed.

Ovarian tumors are named according to the type of cells the tumor started from and whether the tumor is benign or cancerous. The three main types of ovarian tumors are epithelial tumors, germ cell tumors and stromal tumors.

Epithelial ovarian tumors develop from the cells that cover the outer surface of the ovary. Most epithelial ovarian tumors are benign. There are several types of benign epithelial tumors, including serous adenomas, mucinous adenomas, and Brenner tumors. Cancerous epithelial tumors are carcinomas. These are the most common and most deadly of all types of ovarian cancers. There are some ovarian epithelial tumors whose appearance under the microscope does not clearly identify them as cancerous; these are called borderline tumors or tumors of low malignant potential (LMP tumors). Epithelial ovarian carcinomas (EOC's) account for 85 to 90 percent of all cancers of the ovaries.

Ovarian germ cell tumors develop from the cells that produce the ova or eggs. Most germ cell tumors are benign, although some are cancerous and may be life threatening. The most common germ cell malignancies are maturing teratomas, dysgerminomas, and endodermal sinus tumors. Germ cell malignancies occur most often in teenagers and women in their twenties.

Ovarian stromal tumors develop from connective tissue cells that hold the ovary together and those that produce the female hormones, estrogen and progesterone. The most common types among this rare class of ovarian tumors are granulosa-theca tumors and Sertoli-Leydig cell tumors. These tumors are quite rare and are usually considered low-grade cancers, with approximately 70 percent presenting as stage I disease.

Prostate Cancer

Prostate cancer is the most commonly diagnosed cancer in men in the United States and is the second leading cause of cancer-related death in men following lung cancer. There are approximately 200,000 new cases of prostate cancer diagnosed annually and approximately 30-40,000 deaths annually from prostate cancer in the U.S.

While cancer of the prostate is extremely common, its exact cause is not known. When prostatic tissue is examined under a microscope either after prostate surgery or at autopsy, cancer is found in 50 percent of men over age 70 and in virtually all men over age 90. Most of these cancers never cause symptoms because they spread very slowly; however, some prostate cancers do grow more aggressively and spread throughout the body. Although fewer than three percent of the men with the disease die of it, prostate cancer is still the second most common cause of cancer death in men.

Experimental Animal Models

Colon adenocarcinoma in rodents induced by the procarcinogen 1, 2-dimethylhydrazine and its metabolite azoxymethane (AOM) is a well-characterized carcinogen-induced tumor because of its morphological similarity to human colon cancer, high reproducibility and relatively short latency period (Shamsuddin, (1986) Human Path. 17:451-453; herein incorporated by reference). This rodent tumor model is similar to human colon adenocarcinoma not only in its morphology (Shamsuddin & Trump, (1981) J. Natl. Cancer Inst. 66:389-401) but also in the genes that are involved in tumorigenesis (Shivapurkar et al., (1995) Cancer Lett. 96:63-70; Takahashi et al., (2000) Carcinogenesis 21:1117-1120).

In addition to chemical carcinogen-induced models of colon cancer in rodents, gene disruption of the catalytic subunits of phosphoinositide-3-OH kinase (PI3-Kγ) (Sasaki et al., (2000) Nature 406:897-902) or the guanosine-binding protein Gai2 (Rudolph et al., (1995) Nat. Genet. 10: 143-50) causes spontaneous colon cancer in rodents. Both of the aforementioned references are incorporated herein by reference. These studies indicate that potential causes other than alterations in the prototypical tumor suppressor genes and oncogenes could be involved in the etiology of human colon cancer.

A number of animal models for oral squamous cell carcinoma have been developed, including rat, mouse and hamster models. A hamster cheek pouch tumor model induced by the carcinogen 7,12-dimethylbenzanthracene remains one of the most common models (Baker (1986) Malignant neoplasms of the oral cavity. In: Otolaryngology-Head and Neck Surgery, Cummings et al. (eds.) pp. 1281-1343. St. Louis, Mo.: CV Mosby), but exhibits a number of differences from human oral cavity tumorigenesis. A recent mouse model using the carcinogen 4-nitroquinoline 1-oxide (4-NQO) has been developed which more closely simulates many aspects of human oral cavity and esophageal carcinogenesis (Tang et al. (2004) Clin. Cancer Res. 10: 301-313; incorporated herein by reference).

An animal model for multiple myeloma has been described (Garrett et al. (1997) Bone 20: 515-520; incorporated herein by reference), which uses a murine myeloma cell line 5TGM1 that causes lesions characteristic of human myeloma when injected into mice. Such lesions include severe osteolysis and the involvement of non-bone organs including liver and kidney. Mice inoculated with cultured 5TGM1 cells predictably and reproducibly develop disease, symptoms of which include the formation of a monoclonal gammopathy and radiologic bone lesions.

A number of animal models for the study of glioma exist, including an intracerebral rat glioma model (Sandstrom et al. (2004) Br. J. Cancer, 91: 1174-1180), and a murine model using injection of dog-derived J3T1 glioma cells (U.S. Pat. No. 6,677,155) (both incorporated herein by reference).

Animal models for the study of non-small cell lung cancer have been previously described, for example, by xenografting human tumors by subcutaneous transplantation of LC-6 human non-small cell lung cancer into BALB/C-nu/nu mice (Tashiro et al. (1989) Cancer Chemother Pharmacol 24, 187; herein incorporated by reference).

An animal model for the study of stomach cancer has been described which uses AZ-521 human stomach cancer xenografts in nude mice (Fukushima et. al. (2000) Biochem. Pharmacol. 59, 1227-1236; incorporated herein by reference).

Numerous animal models of AML have been previously described, including in rats (Blatt, J et al. (1991) Leuk Res 15:391-394), and SCID mice (Vey, N. et al. (2000) Clin. Cancer Res., 6:731-736) (both incorporated herein by reference).

A number of animal models used for the study of HCC have been described (Chisari et al., (1985) Science 230: 1157-1160; Babinet et al. (1985) Science 230: 1160-11; U.S. patent application Ser. No. 10/439,214) (all incorporated herein by reference). These references disclose the generation of transgenic mouse models of HCC by incorporating the HBV virus into the genome.

Animal models with experimental metastasis pattern resembling those frequently observed in human patients (Engebraaten & Fodstad, (1999) Int J Cancer. 82:219-25; incorporated herein by reference), which use MA-11 and MT-1, two estrogen and progesterone receptor-negative human breast cancer cell lines. Other models for breast cancer include U.S. patent application Ser. No. 10/410,207 (herein incorporated by reference). Alternatively, the ability of the compounds of the present invention to function as anti-breast cancer agents, either alone or in combination with other agents, can be demonstrated in vivo in carcinogen induced mammary tumors in wild type Sprague-Dawley Rats (Thompson H. J et al, Carcinogenesis, (1992) 13:1535-1539; incorporated herein by reference).

A number of animal models for ovarian cancer are known in the art. For example, Connolly et al. ((2003) Cancer Research, 63, 1389-1397; incorporated herein by reference), discloses methods of developing epithelial ovarian cancer in mice by chimeric expression of the SV40 Tag under control of the MISIIR promoter. In another example, Liu et al. (Cancer Research 64, 1655-1663 (2004); incorporated herein by reference) have introduced human HRAS or KRAS oncogenes into immortalized human ovarian surface epithelial cells, which form subcutaneous tumors after injection into immunocompromised mice.

Numerous animal models for the study of prostate cancer are available. One murine model, using prostate cancer xenografts introduced into SCID mice, is disclosed in U.S. Pat. No. 6,756,036 (incorporated herein by reference). Alternatively, an orthotopic mouse model of metastatic prostate cancer can be used, as disclosed in U.S. patent application Ser. No. 10/417,727 (incorporated herein by reference).

24-Hydroxylase Inhibitors

Vitamin D and its analogues are potent regulators of cell growth and differentiation both in vivo and in vitro. Vitamin D2 and Vitamin D3 are ingested through dietary intake. Vitamin D2 is converted to D3 in the skin following exposure to ultraviolet radiation such as sunlight. Vitamin D3 (also called cholecalciferol) is photosynthesized from 7-dehydroxycholesterol (previtamin D3) in skin by UV-induced cleavage of the carbon-carbon bond between C9 and C10, enters circulation, and binds to vitamin D binding protein (DBP) for transport. DBP-bound vitamin D3 is biologically inert and requires activation.

In the liver vitamin D3 is hydroxylated, by the Vitamin D metabolizing enzyme 25-hydroxylase (250Hase) at the C-25 position by a cytochrome P-450 enzyme system (CYP27) to monohydroxyvitamin D₃, 25(OH)D₃, the major circulating form of vitamin D. This metabolite is hydroxylated again, by the Vitamin D metabolizing enzyme 1α-hydroxylase (1αOHase) in the kidney and other organs at the C-1 position by a cytochrome P-450 enzyme reaction (CYP27B1) to form dihydroxyvitamin D₃, 1,25(OH)₂ D₃, also known as calcitriol, the hormonally active vitamin D metabolite.

(OH)D₃ and 1,25(OH)₂ D₃ are metabolized by 24-hydroxylase at C-24 position by a cytochrome P-450 enzyme system (CYP24) to form metabolites 24,25(OH)₂D₃ and 1,24,25(OH)₃ D₃, respectively. These metabolites have been considered inactivation products, but some studies have shown that vitamin D metabolites may have specific effects in target cells such as cellular proliferation.

Calcitriol is a steroid hormone. It plays an important regulatory role in switching cells from proliferation towards differentiation, in calcium homeostasis and immune regulation.

The cellular receptor for calcitriol (designated VDR, for vitamin D receptor) is a member of family II of the hormone receptor superfamily of transcription factors. VDR has been fully characterized and is primarily localized in the nuclear compartment of the cell. In the cell nucleus, VDR, in the presence of calcitriol, heterodimerizes with the retinoid X receptor (RXR). This dimeric complex binds to a vitamin D responsive element (VDRE, characterized by direct repeats of the hexamer AGGTCA spaced by three nucleotides) and activates transcriptions of regulated genes. Among the regulated genes, activation of calcitriol leads to: 1. upregulation of VDR (increasing functional activities of calcitriol), 2. downregulation of CYP27B1 (thus reducing further formation of calcitriol), and 3. upregulation of CYP24 (thus catabolizing calcitriol). As such calcitriol autoregulates its own production and catabolism.

Inhibition of the vitamin D metabolizing enzyme 24-hydroxylase (24OHase) at the C-24 position would be expected to increase levels of intracellular calcitriol and reduce levels of 24-hydroxylated vitamin D metabolites.

In view of the above, novel potent and selective 24-hydroxylase inhibitors are needed to partially or totally inhibit formation of, or otherwise treat (e.g., reverse or inhibit the further development of) cancer such as tumors, neoplasms, carcinomas, sarcomas, leukemias, lymphomas and the like.

An “inhibitor of 24-hydroxylase” is any chemical compound that has the property of reducing the enzyme activity of CYP24, also known as “vitamin D 24-hydroxylase” or “24-hydroxylase.” Important physiological substrates for this enzyme, which is normally found in the inner mitochondrial membrane of proximal renal tubule cells, epidermal keratinocytes, and other cells, are 1,25-(OH)₂ vitamin D₃ and 25-OH vitamin D₃, which it converts to the less active metabolites 1,24,25-(OH)₃ vitamin D₃ and 24,25-(OH)₂ vitamin D₃, respectively. An “inhibitor of 24-hydroxylase” can reduce the rate of the enzyme reaction catalyzed by 24-hydroxylase by any amount, for example, by a statistically significant amount, by at least 1%, at least 2%, at least 3%, at least 5%, at least 10%, at least 15%, at least 20%, at least 30%, at least 50%, at least 100%, at least 2-fold, at least 3-fold, at least 5-fold, at least 10-fold, at least 100-fold, or at least 1000-fold or more. Inhibition can be by any mechanism, for example, by competitive, uncompetitive, or noncompetitive inhibition.

Azoles are potent inhibitors of cytochrome P450 enzymes which directly bind to heme iron via a single electron pair from the azole nitrogen. Further, azoles interact with the substrate binding pocket. See Poulos, Pharm. Res. 5:67-75 (1988). Thus, azole inhibitors of CYP enzymes can block both oxygen and substrate binding and provide high-affinity binding. Examples of azole drugs are the antifungals ketoconazole, clotrimazole, itraconazole, and fluconazole. While these are potent CYP inhibitors, they may not possess adequate selectivity if they are capable of binding to heme iron in different CYP enzymes.

As used herein, an “azole” is a compound comprising a five-membered heterocyclic ring with two double bonds, which ring also contains an atom of nitrogen and at least one other noncarbon atom, such as oxygen, sulphur, or another nitrogen atom. Preferred azoles for use in the invention are those which are capable of inhibiting CYP24, or 24-hydroxylase. More preferred are azole compounds which selectively inhibit CYP24, i.e., compounds which have a lower IC₅₀ value for CYP24 than for other enzymes, including CYP27B, which is responsible for the final step in the synthesis of 1,25-(OH)₂ vitamin D₃. Preferred azoles of the invention are those which have a bulky group attached to the C atom which is alpha to the azole group. A “bulky group” in this context is a cyclic or branched alkyl substituent. For example, the bulky group can be a phenyl, naphthyl, thienyl or pyridyl substituent; or a phenyl, naphthyl, thienyl or pyridyl substituent monosubstituted by halogen, (C₁₋₄)alkoxy, (C₁₋₄)alkyl, di-(C₁₋₄) alkylamino or cyano.

Schuster et al., J. Cell. Biochem. 88:372-380 (2003) (hereby incorporated by reference in its entirety) have determined structure-activity relationships for selective and potent 24-hydroxylase inhibitors. Pharmacophore models were built by superimposing a large group of inhibitors of CYP24 and CYP27B1. A program called DISCO (DIStance COmparison, Tripos), a module of the computational SYBYL software, was used to obtain information on the shape, size, and electrostatic properties of the active site of CYP24. Schuster et al. determined that selectivity for CYP24 was achieved by positioning bulky substituents in the α-position relative to the azole. On the other hand, bulky substituents in the β-position to the azole favored selectivity for CYP27B1. The active sites of both CYP24 and CYP27B1 shared several common features, including a similar large size and the presence of at least two hydrophobic regions. The location of the hydrophobic regions was different, which led to the principle that substitution with large bulky groups in the α-position to the azole favors CYP24 binding whereas large bulky groups in β-position to the azole favors binding to CYP27B1.

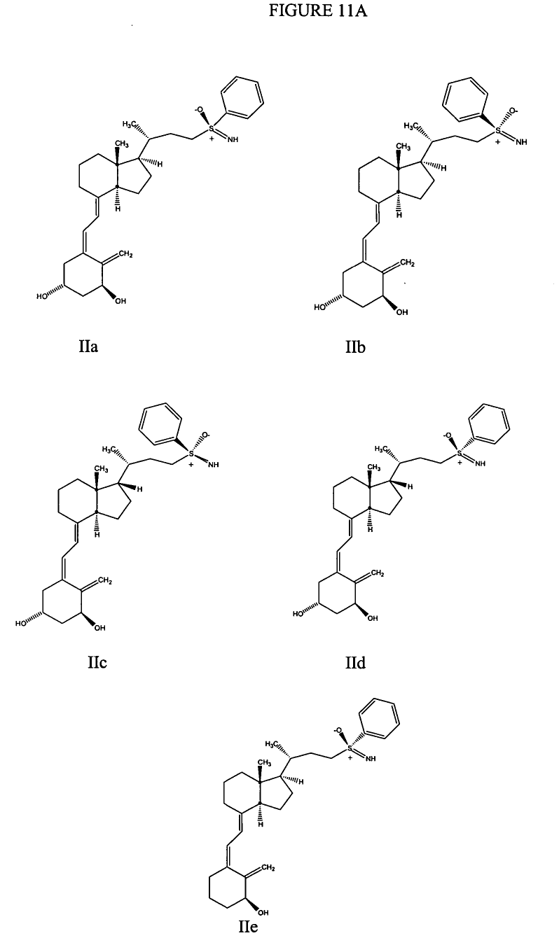

Several specific compounds identified by Schuster et al., J. Cell. Biochem. 88:372-380 (2003) are potent inhibitors of 24-hydroxylase. These include (R)-SDZ-286907, (R)-SDZ-287871, (R)-VAB636, (S)-SDZ-285428, and (R)-VID400 (2-(R)-4′-Chlorobiphenyl-4-carboxylic acid (2-imidazol-1-yl-2-phenyl-ethyl)-amide) (see FIG. 10).

In a specific embodiment, the 24-hydroxylase inhibitor compounds are represented by the structural Formula I or a pharmaceutically acceptable salt, solvate or hydrate thereof, 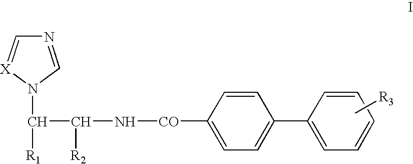
wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄)alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

The acylated aminoalkanimidazoles and aminoalkantriazoles of Formula I are fully described in U.S. Pat. No. 5,622,982 to Schuster et al., the entire content of which is hereby incorporated by reference.

In another embodiment, the 24-hydroxylase inhibitor is represented by the structural Formula II or a pharmaceutically acceptable salt, solvate or hydrate thereof, 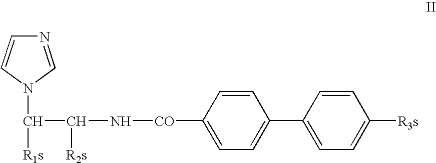
wherein:
R₁ₛ is phenyl, phenyl monosubstituted by halogen, or 1-naphtyl, and R₂ₛ is hydrogen; or
R₁ s is hydrogen and R₂ₛ is pyridyl or 2-(5-chloro)pyridyl; and
R₃ₛ is halogen, (C₁₋₄) alkoxy.

The compounds of Formula II are fully described in U.S. Pat. No. 5,622,982 to Schuster et al., the entire content of which is hereby incorporated by reference.

Vitamin D analogs also can be effective inhibitors of 24-hydroxylase. The structure of 1,25-(OH)₂ vitamin D₃, also known as calcitriol, is depicted below.

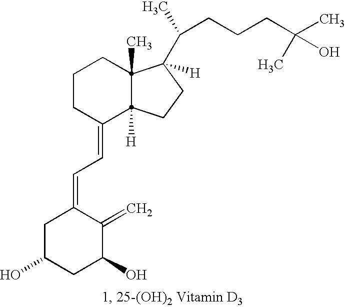

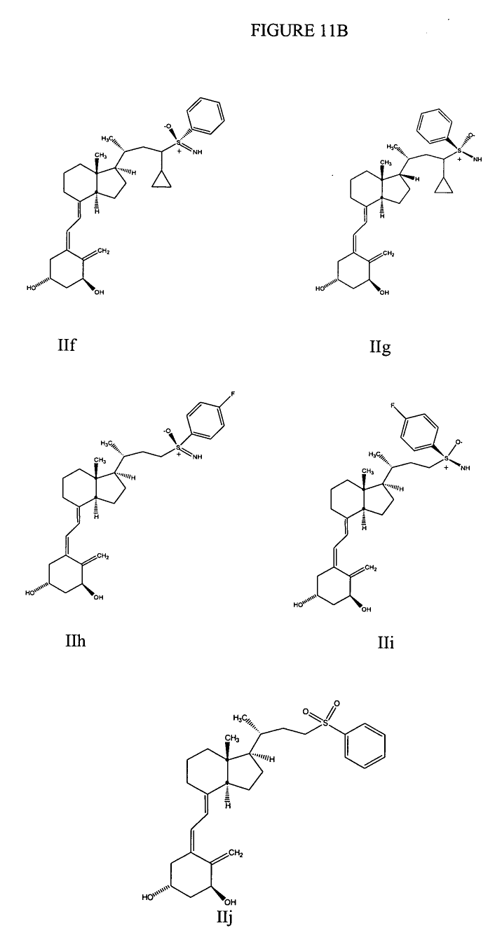

As used herein, a “structural analog of 1,25-(OH)₂ vitamin D₃” is a compound that retains the ring structures and backbone of 1,25-(OH)₂ vitamin D₃, but wherein one or more of the substituents attached thereto (e.g., H, OH, CH₂, or CH₃) have been removed or replaced with other substituents, or wherein the terminal C atom of the backbone (C₂₅) has been replaced with one or more substituents. Preferably, structural analogs of 1,25-(OH)₂ vitamin D₃ of the instant invention have the property of inhibiting 24-hydroxylase or are resistant to degradation by 24-hydroxylase. For example, Kahraman et al. (J. Med. Chem. 47:6854-6863 (2004); incorporated herein by reference) have described a set of 24-sulfoximine derivatives of 1,25-(OH)₂ vitamin D₃. Representative examples with good inhibitory potency for 24-hydroxylase are depicted in FIG. 11.

Thus, in certain embodiments the inhibitor is a structural analog of 1,25-(OH)₂ vitamin D₃. For example, the inhibitor may be represented by Formula IV: 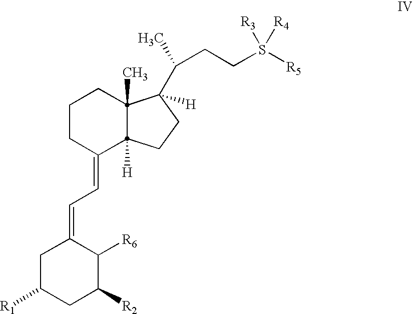
wherein R₁ and R₂ are each independently selected from the group consisting of hydrogen, OR′, —C(O)H, and —C(O)R′;
wherein R′ is selected from the group consisting of a C1 to C₆ alkyl, a cycloalkyl, phenyl, an alkylaryl, an arylalkyl, and a heteroaryl; each of which can be optionally substituted with at least one halogen, thiol, mercapto, hydroxyl, or amino group;
wherein R₃, R₄ and R₅ are each independently selected from the group consisting of hydrogen, hydroxyl, oxy, imine, phenyl, a C1 to C₆ alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R₆ is hydrogen, ═CH₂ or a C₁ to C₆ alkyl, alkenyl, cycloalkyl, or cycloalkenyl, each of which can be optionally substituted with at least one halogen, thiol, mercapto, hydroxyl, or amino group;
   or a pharmaceutically acceptable salt, hydrate, solvate, ester, or isomer thereof.

Structure activity relationships for 24-sulfoximine analogs of 1,25-(OH)₂ vitamin D3 have been described by Kahraman et al. The most potent compound appears to be the phenyl sulfoximine shown in Formula V:

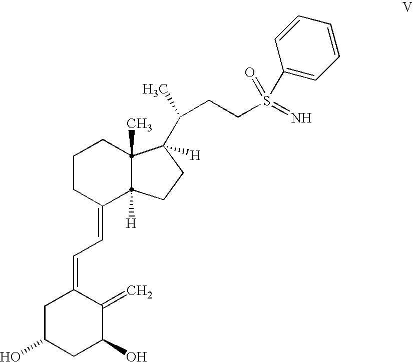

Only slightly less potent is the 4-fluorophenyl sulfoximine shown in Formula VI: 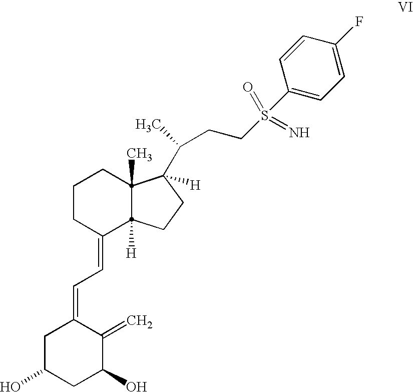
In general, the stereochemical configuration at the 24-sulfur atom is significant, with the 24-(S) configuration being more potent than the 24-(R) configuration. 24-sulfone analogs are less potent that 24-sulfoximines, and 22-ene analogs are much less potent.

Retinoid X Receptor and Retinoid Acid Receptors

There are two main types of retinoid receptors that have been identified in mammals (and other organisms). The two main types or families of receptors are respectively designated the Retinoid Acid Receptors (RARs) and Retinoid X Receptors (RXRs).

The Retinoid X Receptor (RXR) is a member of the nuclear hormone receptor family of proteins. RXR contains two signature domains of nuclear receptor family proteins, the DNA-binding domain and ligand binding domain (LBD). RXR is a ligand-dependent transcription factor. The endogenous ligand for RXR is 9-cis retinoic acid. RXR plays an important role in many fundamental biological processes such as reproduction, cellular differentiation, bone development, hematopoiesis and pattern formation during embryogenesis (Mangelsdorf, D. J. et al., Cell, 83: 841-850 (1995)).

The mammalian RXR includes at least three distinct genes, RXRα, RXRβ and RXRγ (RXR alpha, beta and gamma) which give rise to a large number of protein products through differential promoter usage and alternative splicing. Besides acting as a homodimer, RXR plays a central role in regulating the activity of other nuclear hormone receptors by acting as a partner for heterodimers. RXR forms a functional heterodimer with retinoic acid receptor (RAR), vitamin D receptor, and many other nuclear receptors. RAR exists as three major subtypes: RARα, RARβ and RARγ (RAR alpha, beta and gamma). The different binding partners of the RXR render a different DNA-binding specificity of the heterodimer.

The invention further relates to pharmaceutical composition useful for the treatment of cancer comprising a 24-hydroxylase inhibitor. In a particular embodiment, the pharmaceutical composition further comprises calcitriol. The 24-hydroxylase inhibitor and the calcitriol can each be present in the pharmaceutical composition in a therapeutically effective amount. In another aspect, the 24-hydroxylase inhibitor and the calcitriol together comprise a therapeutically effective amount. The pharmaceutical composition of the present invention can optionally contain a pharmaceutically acceptable carrier.

The invention further relates to use of a 24-hydroxylase inhibitor for the manufacture of a medicament for treating cancer.

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In a preferred embodiment, the mammal is a human.

As used herein, treating and treatment refer to partially or totally inhibiting formation of, or otherwise treating (e.g., reversing or inhibiting the further development of) cancer such as tumors, neoplasms, carcinomas, sarcomas, leukemias, lymphomas and the like.

As used herein, therapeutically effective amount refers to an amount sufficient to elicit the desired biological response. In the present invention, the desired biological response is partially or totally inhibiting formation of, or otherwise treating (e.g., reversing or inhibiting the further development of) cancer such as tumors, neoplasms, carcinomas, sarcomas, leukemias, lymphomas and the like.

The therapeutically effective amount or dose will depend on the age, sex and weight of the patient, and the current medical condition of the patient. The skilled artisan will be able to determine appropriate dosages depending on these and other factors to achieve the desired biological response. A typical dosage for a human is in the range of 0.001 to 100 mg/kg/day, preferably 0.01 to 10 mg/kg/day or 0.1 to 1 mg/kg/day. Suitable dosing ranges for 24-hydroxylase inhibitors can be, for example, from about 100 micrograms to about 2 g per day, for example, from about 200 micrograms to about 1 g per day, such as from about 300 micrograms to about 750 mg per day, or for example, from about 400 micrograms to about 600 mg per day.

Modes of Administration

The compounds for use in the method of the invention can be formulated for administration by any suitable route, such as for oral or parenteral, for example, transdermal, transmucosal (e.g., sublingual, lingual, (trans)buccal), vaginal (e.g., trans- and perivaginally), (intra)nasal and (trans)rectal), subcutaneous, intramuscular, intradermal, intra-arterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays, dry powders or aerosolized formulations.

It is preferred that the compounds are orally administered. Suitable oral dosage forms include, for example, tablets, capsules or caplets prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated, e.g., to provide for ease of swallowing or to provide a delayed release of active, using suitable methods. Liquid preparation for oral administration can be in the form of solutions, syrups or suspensions. Liquid preparations (e.g., solutions, suspensions and syrups) are also suitable for oral administration and can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

As used herein, the term pharmaceutically acceptable salt refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

The 24-hydroxylase inhibitor compounds disclosed can be prepared in the form of their hydrates, such as hemihydrate, monohydrate, dihydrate, trihydrate, tetrahydrate and the like and as solvates.

It is understood that 24-hydroxylase inhibitor compounds can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

Combination Administration

In a particular embodiment, the 24-hydroxylase inhibitor is co-administered with calcitriol. Administration of a 24-hydroxylase inhibitor can take place prior to calcitriol treatment, after the calcitriol treatment, at the same time as the calcitriol or a combination thereof. The calcitriol can be administered prior to onset of treatment with the 24-hydroxylase inhibitor or following treatment with the 24-hydroxylase inhibitor. In addition, calcitriol treatment can be administered during the period of 24-hydroxylase inhibitor administration but does not need to occur over the entire 24-hydroxylase inhibitor treatment period.

Effective amounts of calcitriol are well known in the art. In some embodiments, a 24-hydroxylase inhibitor and calcitriol are each administered in an amount which alone does not provide a therapeutic effect (a sub-therapeutic dose). In yet another embodiment, the 24-hydroxylase inhibitor can be administered in a therapeutically effective amount, while calcitriol is administered in a sub-therapeutic dose. In still another embodiment, the 24-hydroxylase inhibitor can be administered in a sub-therapeutic dose, while calcitriol is administered in a therapeutically effective amount. In general, the ratio of the 24-hydroxylase inhibitor calcitriol, in terms of the therapeutically effective dose of each drug given alone, can be varied from at least 1000/1 to 1/1000 by weight. The ratio of 24-hydroxylase inhibitor to calcitriol can be, for example, in the range of about 1:1000, 1:100, 1:50, 1:10, 1:1, 10:1, 50:1, 100:1, or 1000:1 on a weight basis. It is understood that the method of coadministration of a first amount of a 24-hydroxylase inhibitor and a second amount of calcitriol can result in an enhanced or synergistic therapeutic effect, wherein the combined effect is greater than the additive effect that would result from separate administration of the first amount of the 24-hydroxylase inhibitor and the second amount of calcitriol. A synergistic effect can be, for example, an increase of 3-fold, 10-fold, 100-fold or greater therapeutic effect than the sum of the therapeutic effects expected from administering each agent separately. The greater therapeutic effect can be manifested in a variety of ways, for example, greater reduction in tumor size, more rapid reduction in tumor size, reduced morbidity or mortality, or longer time until recurrance of the tumor. Where synergistic effects are encountered, the dosage of each individual drug in the combination can be varied so as to achieve the desired effect.

Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When compounds of the present invention contain one chiral center, the compounds exist in two enantiomeric forms and the present invention includes either or both enantiomers and mixtures of enantiomers, such as the specific 50:50 mixture referred to as a racemic mixture. The enantiomers can be resolved by methods known to those skilled in the art, for example by formation of diastereoisomeric salts which may be separated, for example, by crystallization (See, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least about 99% enantiomeric excess.

When a compound of the present invention has two or more chiral carbons, it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

Experimental Methods

Cell Culture

The human ovarian adenocarcinoma cell line, OVCAR-3 (ATCC, Manassas, Va.) was maintained, as recommended by the supplier, in RPMI 1640 medium (Sigma Aldrich, St. Louis, Mo.) supplemented with 10% FBS, 10 μg/ml insulin, 0.25% glucose and antibiotics (100 IU/ml penicillin, 100 μg/ml streptomycin). Human ovarian adenocarcinoma cell lines UT-OC-1, UT-OC-2, UT-OC-3, UT-OC-4, UT-OC-5 (Grenman, S., Engblom, P., Rantanen, V., Klemi, P. and Isola, J., “Cytogenetic Characterization of Five New Ovarian Carcinoma Cell Lines,” Acta Obstet. Gynecol. Scand., 76:83 (1997)) and SK-OV-3 and a human keratinocyte cell line HaCaT were grown in DMEM (Sigma Aldrich) with 10% FBS and antibiotics (100 IU/ml penicillin, 100 μg/ml streptomycin), and monkey kidney COS cells were maintained in DMEM/F12 (Sigma Aldrich) with 5% FBS. All cell lines were kept at 37° C. in a humidified 95% air/5% CO₂ incubator.

Experiment I

Cell Growth Assay

When OVCAR-3 cells were on the logarithmic growth phase (70% confluent) the growth assay was started. For cell growth assay, 2,000 cell/200 μl/well were plated on 96-well culture plates. One day after plating, the medium (RPMI 1640, [Sigma Aldrich] supplemented with 10% FBS, 10 μg/ml insulin, 0.25% glucose and antibiotics) was changed and indicated concentrations of 1,25(OH)₂D₃, 25(OH)D₃, EB 1089 (Leo Pharmaceutical Products, Ballerup, Denmark), VID400 or combination of VID400 and 1,25(OH)₂D₃ or 25(OH)D₃ were added (day 0). Ethanol was used as a vehicle, and it was also included in the control. The medium containing ethanol vehicle and/or hormones were changed to a fresh one every third day. Cell growth samples were taken 0, 1, 3, 5, 7, 9 and 11 days after the treatment. Preliminary studies showed that during this period cells were at a logarithmic growth phase.

Relative cell numbers were quantified as described previously (Kueng W, Silber E, Eppenberger U. Quantification of cells cultured on 96-well plates. Anal. Biochem., 182:16-9 (1989)). Cells were fixed on the bottom of the wells by addition of 10 μl of 11% glutaraldehyde solution in 0.1% phosphate buffer to 100 μl of medium. The plate was shaken 500 cycles/min for 15 min, washed 3 times by submersion in de-ionised water and air-dried. Fixed cells were stained with 0.1% solution of crystal violet dissolved in de-ionised water. After 20 min incubation, excess dye was removed by carefully washing with de-ionised water. The plate was air-dried prior to a bound dye solubilisation in 100 μl of 10% acetic acid. Relative cell number was given as absorbance units by measuring the optical density (590 nm) from each well using Victor 1420 multilabel counter (Wallac, Turku, Finland). Six determinations were used to calculate the mean optical density ±SD in each concentration at each time point. The absorbance value of day 0 (an overnight culture of 2,000 cells/well) was set as 0 by subtracting it from each value obtained from adjacent time-point measurements (days 1-11), and based on these values growth curves were created. Experiments were repeated 3-5 times. Day 11 was used to compare the effect of hormone treatments and 24OHase inhibitor. Statistical analyses were done using Student's t-test.

Regulation of OVCAR-3 Cell Growth by Different Vitamin D Compounds

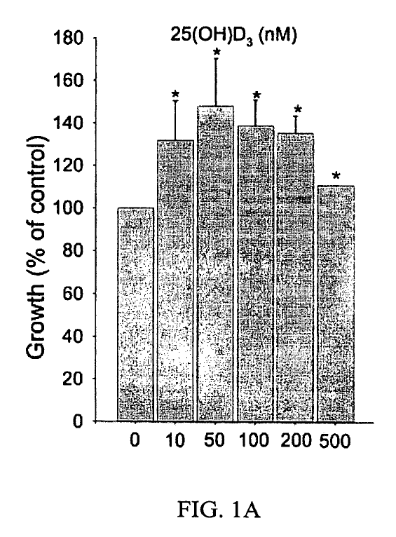

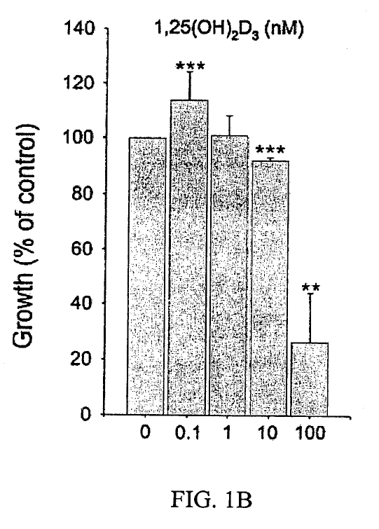

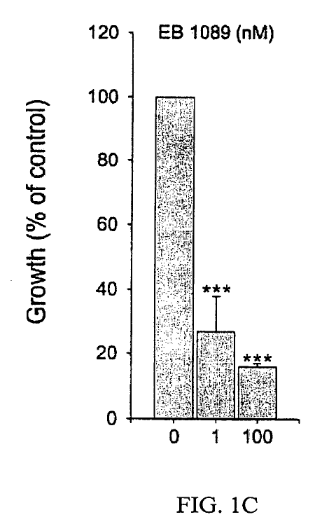

FIG. 1A illustrates the concentration-dependent stimulation of the cell growth with 25(OH)D3 in OVCAR-3 cell line. An amount of 10 nM 25(OH)D3 treatment stimulated growth by 32%, 50 nM stimulated growth by 41%, 100 nM by 39%, 200 nM by 35% and 500 nM 25(OH)D₃ by 11% when compared to the control (FIG. 1A). All differences were statistically significant when compared to the control (p<0.05).

When high concentrations were used, 1,25(OH)₂D₃ inhibited growth of the OVCAR-3 cell line (FIG. 1B). An amount of 100 nM 1,25(OH)₂D₃ inhibited growth by 74% (p<0.001) and 10 nM by 8% (p<0.0001) when compared to the control. An amount of 0.1 nM 1,25(OH)₂D₃ stimulated growth by 14% (p<0.0001),whereas 1 nM 1,25(OH)₂D₃ did not have an effect on the cell growth.

EB 1089 inhibited growth when 1 and 100 nM concentrations were used (FIG. 1C). When compared to the control, 100 nM EB 1089 inhibited growth by 84% and 1 nM by 73% (p<0.0001). At 1 nM concentration, EB 1089 was as potent a growth inhibitor as 100 nM EB 1089. The growth inhibition was almost equal to 100 nM 1,25(OH)₂D₃ and 1 nM EB 1089 (74% vs. 73% of the control).

Experiment II

Detection of 24- and 1α-Hydroxylase mRNAs

To test whether enzymes 1α-hydroxylase and 24-hydroxylase are involved in the metabolism of vitamin D compounds in the OVCAR-3 cell line, we studied the expression of these enzymes at mRNA level. We also studied whether the expression of 24OHase mRNA could be modulated by 25(OH)D₃, 1,25(OH)₂D₃ or EB 1089.

When cell culture bottles were grown to 70% confluence, the old medium was removed and replaced with medium containing 100 nM 1,25(OH)₂D3, 25(OH)D3 or EB 1089. Ethanol was used as a vehicle, and it was also added to the control cells. For RNA extraction, the cells were collected 4, 6 and 24 hr after the treatment with vitamin D compound or vehicle. RNA extractions were done with TRIZOL reagent (GIBCO Invitrogen Corporation, Paisley, UK). The integrity of RNA samples was confirmed on gel electrophoresis.

The expression of 24- and 1αOHase messenger RNA was detected using a reverse transcription-polymerase chain reaction (RT-PCR). To perform the RT-PCR, specific oligonucleotide primers were synthesised by Amersham Bioscience (Amersham, UK) (Table I).

TABLE I
OLIGONUCLEOTIDE PRIMER SEQUENCES FOR RT-PCR

| Gene | | | | Product |
|---|---|---|---|---|
| (accession | Base | Oli- | | Size |
| no.) | pairs | gos | Sequence | (bp) |
| 1αOHase | 1241-1261 | F | 5′-GTCAAGGAAGCTA | 303 |
| | | | AGACTG-3′ | |
| | | | (SEQ ID NO: 1) | |
| (AB005038) | 1524-1543 | R | 5′-TGTTAGGATGTGG | |
| | | | GCCAAAG-3′ | |
| | | | (SEQ ID NO: 2) | |
| 24OHase | 833-852 | F | 5′-TGATCCTGGAAGG | 212 |
| | | | GGAAGAC-3′ | |
| | | | (SEQ ID NO: 3) | |
| (L13286) | 1023-1044 | R | 5′-CACGAGGCAGATA | |
| | | | CTTTGAAAC-3′ | |
| | | | (SEQ ID NO: 4) | |
| PBGD | 695-714 | F | 5′-AAGTGCGAGCCAA | 298 |
| | | | GGACCAG-3′ | |
| | | | (SEQ ID NO: 5) | |
| (X04808) | 969-992 | R | 5′-TTACGAGCAGTGA | |
| | | | TGGGTAGCAAC-3′ | |
| | | | (SEQ ID NO: 6) | |

F, forward primer; R, reverse primer.

The reactions for 24-hydroxylase were performed in the LightCycler instrument (Roche Diagnostics, Basel, Switzerland) from 300 ng total RNA. PBGD (human porphobilinogendeaminase) mRNA was used as an external control. A master mix of the following components was prepared in a 20 μl volume: 0.5 μM PBGD primers or 0.3 μM 24OHase primers and 3.5 mM Mn²⁺ for PBGD or 3.25 mM Mn²⁺ for 24OHase. Nucleotides, Tth DNA polymerase (DNA polymerase and reverse transcriptase activity), SYBR Green I and reaction buffer were included in the LightCycler-RNA Master SYBR Green I kit (Roche Diagnostics). For preparing the standard curve, total RNA from HaCaT cells, which express 24-hydroxylase mRNA (Harant, H., Spinner, D., Reddy, G S., Lindley, I J., “Natural Metabolites of 1alpha,25-dihydroxyvitamin D(3) Retain Biologic Activity Mediated Through the Vitamin D Receptor, J. Cell. Biochem., 78:112-20 (2000)), was amplified in the same run as samples. The RT-PCR protocol was as follows: 20 min reverse transcription at 61° C. and 30 sec denaturation at 95° C. followed by 45 cycles with a 95° C. denaturation for 1 sec, 62° C. for PBGD or 57° C. for 24OHase annealing for 7 sec and 72° C. extension for 12 sec. Detection of fluorescent product was performed at the end of the extension step of each cycle. To verify the specific products, melting curve analysis and gel electrophoresis were done. The data were quantified by the Fit Points method with LightCycler Data Analysis software. The amplification efficiency and the relative expression ratio of 24OHase were calculated according to Pfaffl, M W., “A New Mathematical Model for Relative Quantification in Real-time RT-PCR,” Nucleic Acids Res., 29:2002-7 (2001). Hormone treatments and RT-PCR were done twice.

A normal RT-PCR was used for the detection of 1αOHase mRNA. RT-PCR=(RobusT RT-PCR Kit, Finnzymes, Espoo, Finland) was performed according to the manufacturer's instructions from 1 μg total RNA. A negative control reaction (reactions without reverse transcriptase enzyme) was done from each sample. The RT-PCR protocol was as follows: 30 min reverse transcription at 48° C. and 2 min denaturation step at 94° C. followed by 30 cycles with 94° C. denaturation for 30 sec, 54° C. annealing for 30 sec and 72° C. extension for 30 sec. The final extension after cycles was at 72° C. for 7 min. Total RNA (0.5 μg) from monkey kidney COS cells transfected with human 1αOHase cDNA (Laboratory of Dr. S. Kato, Institute of Molecular and Cellular Biosciences, University of Tokyo, Japan) was used as a positive control. A transfection was done according to the manufacturer's instructions with 1αOHase ORF cDNA in pcDNA3 mammalian expression vector by a lipofection (Lipofectamine, Life Technologies). A functional control reaction (MS2 RNA and primers for amplification of 1100 bp sequence) was included in the kit, and it was carried out with the same run as other samples. After gel electrophoresis, RT-PCR products were extracted from the gel and sequences were verified by hybridization with ³²P-labelled RNA-probe made from 1αOHase cDNA.

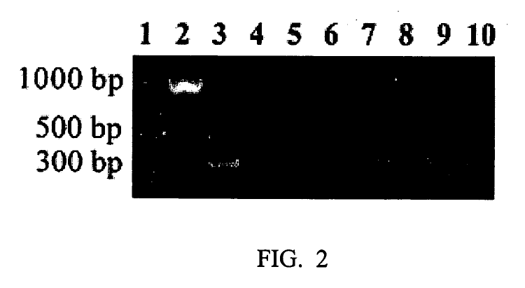

The data indicate that the OVCAR-3 cell line expresses 1αOHase (FIG. 2). A single 303 bp band can be seen in 1αOHase transfected COS sample (lane 3) and in both ethanol-treated control (lanes 7 and 8) and 100 nM 1,25(OH)₂D₃-treated (lanes 9 and 10) OVCAR-3 samples. A hybridisation with P³²-labelled probe showed that the 1αOHase sequence is amplified in RT-PCR. 1αOHase mRNA was also expressed in 6 other ovarian cancer cell lines (UT-OC-1-5 and SK-OV-3; data not shown).

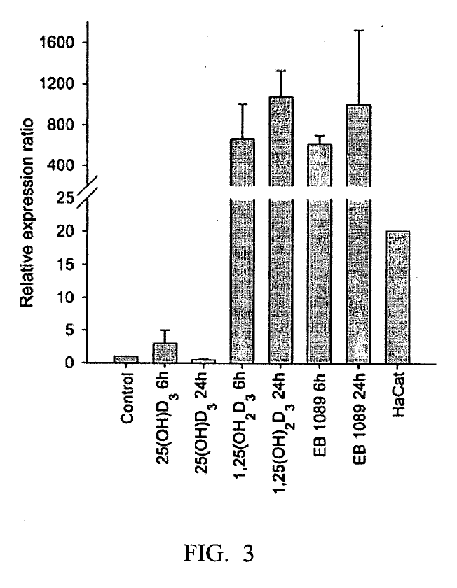

Also, 24OHase is expressed in OVCAR-3 cells and the expression of 24OHase is regulated by EB 1089 and 1,25(OH)₂D₃ almost equally. After 6 hr treatment, the expression of 24OHase mRNA (FIG. 3) was induced 650-fold with 100 nM 1,25(OH)₂D₃ and 600-fold with 100 nM EB 1089. After 24 hr, the expression levels were further increased. When compared to the control, 1,25(OH)₂D₃ treatment induced the expression by 1,100-fold and EB 1089 by 1,000-fold. After 6 hr treatment, the expression in 25(OH)D₃- (100 nM) treated cells was slightly increased (3-fold) but returned to a basal level or even slightly downregulated (0.5 fold) after 24 hr treatment. The human keratinocyte cell line, HaCaT, was used as a control for the expression of 24OHase, and the data indicate that the basal expression level is 20 times higher in HaCaT than in OVCAR-3 cells.

Experiment III

Metabolic Analysis of 25(OH)D₃

OVCAR-3 cells (1.5×10⁶ cell/flask) were plated on T25 culture flasks. One day after plating, cells were treated with 500 nM 25(OH)D₃ in RPMI 1640 medium supplemented with 10% FBS, 10 μg/ml insulin, 0.25% glucose and antibiotics (100 IU/ml penicillin, 100 μg/ml streptomycin). After 0, 3 or 24 hr, the medium was collected and the cell monolayer was extracted with 1 ml methanol. After 15 min incubation at room temperature, the methanol was transformed into the same tube than the sample medium. The samples for the measurement of the 25(OH)D₃ metabolites were purified using the acetonitrile-C 18 Sep-Pak (Waters Corporation, Milford, Mass.) procedure (Turnbull, H., Trafford, D. J., Makin, H. L., “A Rapid and Simple Method for the Measurement of Plasma 25-hydroxyvitamin D₂ and 25-hydroxyvitamin D₃ Using Sep-Pak C 18 Cartridges and a Single High-Performance Liquid Chromatographic Step,” Clin. Chim. Acta., 120:65-76 (1982)) followed by separation of the metabolites by a high-performance liquid chromatography. The concentrations of 24,25(OH)₂D₃ were quantified by a competitive protein binding assay (Parviainen, M. T., Savolainen K. E., Korhonen, P. H., Alhava, E. M., Visakorpi, J. K., “An Improved Method for Routine Determination of Vitamin D and its Hydroxylated Metabolites in Serum from Children and Adults,” Clin. Chim. Acta, 114:233-47 (1981)) and 1,25(OH)₂D₃ by a radioreceptor assay (Reinhardt, T. A., Horst, R. L., Orf, J. W., Hollis, B. W., “A Microassay for 1,25-dihydroxyvitamin D Not Requiring High Performance Liquid Chromatography: Application to Clinical Studies,” J. Clin. Endocrinol. Metab., 58:91-8 (1984). The second measurement was done following the same procedure except dextran charcoal-treated FBS was used instead of FBS, 24OHase inhibitor (200 nM VID400) was used with the 500 nM 25(OH)D₃ treatment, and the samples were collected only after 0 and 24 hr.

Metabolism of 25(OH)D₃ in OVCAR-3 Cells

The functionality of 24OHase and 1αOHase in the OVCAR-3 cell line was studied. Analysis of metabolites generated from 25(OH)D₃ are shown in Table II. In the first experiment, the amount of 24,25(OH)₂D₃ was 4 times higher after 3 hr incubation than it was when the experiment started (0 hr). After 24 hr, the production was further increased (18-fold). The basal level of 1,25(OH)₂D₃ was 23 pM, and after 3 hr incubation, the concentration was increased to 37 pM. After 24 hr, the concentration was almost equal or slightly decreased (33 pM).

In the second experiment, we supplemented RPMI 1460 medium with dextran charcoal-treated FBS instead of normal FB S. In this experiment, the concentration of 24,25(OH)₂D₃ was increased 27 times after 24 hr. When 24OHase inhibitor was used, the production reduced to one-third when compared to 500 nM 25(OH)D₃ treatment alone. At the beginning of the experiment (0 hr), the concentration of 1,25(OH)₂D₃ was undetectable, but after 24 hr we could detect 28 pM concentration of 1,25(OH)₂D₃. 24OHase inhibitor did not have an effect on production of 1,25(OH)₂D₃.

TABLE II
METABOLITES OF 25(OH)D₃

| | |
|---|---|
| 24,25(OH)₂ | 1,25(OH)₂ |
| D₃ (nM) | D₃ (pM) |

| | I | II¹ | I | II¹ |
|---|---|---|---|---|
| 500 nM 25(OH)D₃, 0 hr | 6 | 1 | 23 | <20 |
| 500 nM 25(OH)D₃, 3 hr | 24 | ns | 37 | ns |
| 500 nM 25(OH)D₃, 24 hr | 112 | 27 | 33 | 28 |
| 500 nM 25(OH)D₃ + | ns | 8 | ns | 27 |
| 200 nM VID400, 24 hr | | | | |

¹Cells were grown in RPMI 1640 supplemented with 10% dextran charcoal-treated FBS instead of FBS.
ns, not studied.

Experiment IV

Effect of 24OHase Inhibitor on Growth Response of 1,25(OH)₂D₃ and 25(OH)D₃

Because the metabolic measurements showed an extensive production of 24,25(OH)₂D₃ and an enzymatic activity of 24OHase, the effect of the 24OHase inhibitor, VID400, on the growth response of 1,25(OH)₂D₃ and 25(OH)D₃ was tested. The cell growth response was determined as described in Experiment I. Briefly, cells were treated with indicated hormone concentrations or combinations of hormone and 24OHase inhibitor (VID400) for 11 days. The growth medium and hormones were changed to a fresh one every third day. After the treatment period, cells were fixed, stained with crystal violet, and the optical density (590 nm) was determined. The cell growth is presented as a percentage of ethanol-treated cells. The values represent the mean of 3 separate experiments ±SD. (*p<0.05, **p<0.001, ***p<0.0001, Student's t-test).

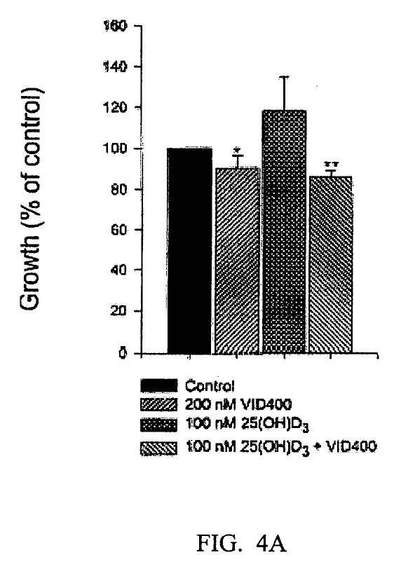

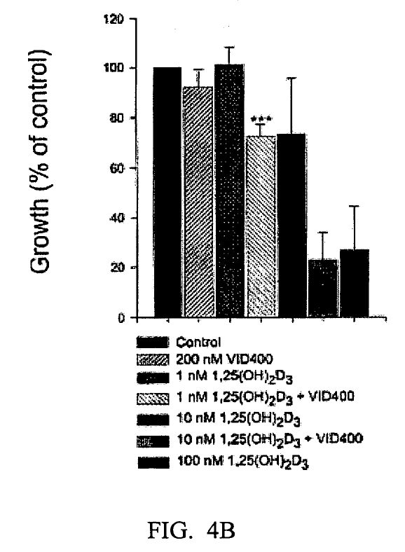

As shown in FIGS. 4A and 4B, 200 nM VID400 alone had a growth-inhibitory effect on cells. The inhibition was 8% (p<0.05) when compared to the control. In these experiments, 100 nM 25(OH)D₃ stimulated growth by 18% (FIG. 4A), but the difference was not statistically significant when compared to the control. When 100 nM 25(OH)D₃ was combined with 200 nM VID400, the stimulatory growth effect was converted to an inhibitory growth effect (14%,p<0.001 when compared to the control).

The effect of 24OHase inhibitor on the growth response of 1,25(OH)₂D₃ (FIG. 4B) was also studied. In these experiments, 1 nM 1,25(OH)₂D₃ alone did not have an effect on the cell growth. However, when it was combined with 200 nM VID400, it inhibited the growth by 27% (p<0.0001) when compared to the control. An amount of 10 nM 1,25(OH)₂D₃ alone inhibited the growth by 26%, but a combination of 10 nM 1,25(OH)₂D₃ and 200 nM VID400 inhibited growth by 77%.

Experiment V

Nuclear Receptors

The sensitivity of seven human ovarian cancer cell lines SK-OV-3, OVCAR-3, UT-OC-1, UT-OC-2, UT-OC-3, UT-OC-4 and UT-OC-5 to 1,25(OH)₂D₃, EB 1089, all-trans-retinoic acid (ATRA) and 9-cis retinoic acid (9-CRA) was studied by evaluating the expression of the vitamin D receptor (VDR), retinoic acid receptor (RAR), retinoic X receptor (RXR) and nuclear receptor coregulators in the cell lines.

Cell Growth Assay

The cell growth assay was conducted according to the procedure described in Experiment I, except that one day after plating the medium was changed and appropriate concentrations of 1,25(OH)₂D₃, EB 1098 (Leo Pharmaceutical Products, Ballerup, Denmark), 9-CRA, ATRA (ICN Biomedicals Inc., Aurora, OHIO), VID 400 (specific 24OHase inhibitor, Novartis Research Institute, Vienna, Austria) or the combinations of VID400 and 1,25(OH)₂D₃, 9-cis retinoic acid (9-CRA) or all-trans-retinoic acid (ATRA) were added (day 0). Cell growth samples were obtained after 11 days treatment.

Ribonuclease Protection Assay

The ribonuclease protection assay (RPA) was used to detect mRNAs of different nuclear receptors and cofactors in SK-OV-3, OVCAR-3, UT-OC-1, UT-OC-2, UT-OC-3, UT-OC-4 and UT-OC-5 cell lines treated for 24 h with 100 nM 1,25(OH)₂D₃, 100 nM EB 1089, 10 μM 9-CRA, 10 μM ATRA or vehicle. After the treatment, RNA was extracted with TRIzol reagent (Invitrogen Life Technologies, Paisley, Scotland, UK). The RPA method and probe sets are previously described (Vienonen, A., Miettinen, S., Manninen, T., Altucci, L., Wilhelm, E., and Ylikomi, T., “Regulation of Nuclear Receptor and Cofactor Expression in Breast Cancer Cell Lines, Eur. J. Endocrinol., 148: 469-479 (2003)). Briefly, ³²P-labelled ([α-³²P]UTP, Amersham Biosciences, Buckinghamshire, UK) RNA-probes were synthesised using in vitro transcription reaction (In vitro transcription reaction kit, Pharmingen, San Diego, Calif.) using two different template sets. The VDR template set generates probes for VDR (326 bp), RXRα (289 bp), RXRβ (258 bp), RXRγ (202 bp), RARα (166 bp), RARβ (182 bp) and RARγ (202 bp). The probe for 24OHase (212 bp, Gene Bank Ac# L13286, bp 833-1044) was included in this set. A coregulator set produces probes for NCoR (360 bp), SMRT (310 bp), pCAF (267 bp), CBP (234 bp), TIF2 (200 bp), AIB1 (179 bp), SRC-1a (145 bp) and −1e (160 bp) and p300 (127 bp); 18S (80 bp) was used as loading control with each probe set. RPA (RPA III, Ambion, Austin, Tex., USA) was done according to the manufacturer's instructions. ³²P-labelled RNA-probes (10⁶ cpm/sample) were hybridized with 8 μg total RNA samples from cells treated for 24 hours with 100 nM 1,25(OH)₂D₃, 100 nM EB 1089, 10 μM 9-CRA, 10 μM ATRA or ethanol. A molar excess of the probes was verified with a 32 μg RNA sample. After overnight hybridization single-stranded RNA was digested with RNase and double-stranded hybridization, products of different lengths were separated by gel electrophoresis. An intensifying screen was exposed and scanned (Storm, Molecular Dynamics, Amersham Biosciences, Buckinghamshire, UK). The results were obtained using computer program ImageQuant 5.1 (Molecular Dynamics, Amersham Biosciences, Buckinghamshire, UK). MCF-7 cells were used as control for cofactor expressions (Vienonen, A., Miettinen, S., Manninen, T., Altucci, L., Wilhelm, E., and Ylikomi, T., “Regulation of Nuclear Receptor and Cofactor Expression in Breast Cancer Cell Lines, Eur. J. Endocrinol., 148: 469-479 (2003)).

cDNA Synthesis and Quantitative Real-Time PCR

Quantitative real-time PCR was used to verify the RPA results and quantify mRNAs whose expressions were too low for the RPA method. For VDR (NM_{—}000376) amplification, the forward primer was 5′-CCTTCACCATGGACGACATG-3′ (SEQ ID NO: 7), corresponding to base 948-967, and the reverse primer 5′-CGGCTTTGGTCACGTCACT-3′ (SEQ ID NO: 8) (base 1025-1007). For RARα (X06538) amplification, the forward primer was 5′-AGTACTGCCGACTGCAGAAGTG-3′ (SEQ ID NO: 9) (base 648-669), the reverse primer 5′-TGTTTCGGTCGTTTCTCACAGA-3′ (SEQ ID NO: 10) (base 695-716). For RARβ (X07282) amplification, the forward primer was 5′-CAAATCATCAGGGTACCACTATGG-3′ (SEQ ID NO: 11) (base 601-624) and the reverse primer 5′-CTGAATACTTCTGCGGAAAAAGC-3′ (SEQ ID NO: 12) (base 651-673). For RARγ (M24857) amplification, the forward primer was 5′-TGCCGGCTACAGAAGTGCTT-3′ (SEQ ID NO: 13) (base 847-866), the reverse primer being 5′-CTTCTTGTTCCGGTCATTTCG-3′ (SEQ ID NO: 14) (base 895-915). For RXRβ (M84820) amplification, the forward primer was 5′-AGCAGCAGGGACGGTTTG-3′ (SEQ ID NO: 15) (base 1559-1576) and the reverse primer was 5′-GATGCTCTAGACACTTAAGGCCAAT-3′ (SEQ ID NO: 16) (base 1612-1636). For RXRγ (U38480) amplification, the forward primer was 5′-TTTCCCGCAGGCTATGGA-3′ (SEQ ID NO: 17) (base 58-75), the reverse primer 5′-TGCTGATGGGCTCATGGAT-3′ (SEQ ID NO: 18) (base 102-120). The primers for 24OHase and RPLP0 (acidic ribosomal phosphoprotein P0) and the procedures for cDNA synthesis and quantitative real-time PCR have been earlier described (Lou, Y. R., Laaksi, I., Syvala, H., Blauer, M., Tammela, T. L., Ylikomi, T., and Tuohimaa, P., “25-Hydroxyvitamin D3 is an Active Hormone in Human Primary Prostatic Stromal Cells,” FASEB J, 18: 332-334 (2004)). Briefly, primer pairs (TAG Copenhagen A/S, Copenhagen, Denmark) were selected in different exons in order to detect amplification from genomic DNA. RPLP0 was used as reference gene. The reverse transcriptase reaction was elicited using the High Capacity cDNA Archive Kit (Applied Biosystems, Foster City, Calif.) and the real-time PCR step using SYBR® Green PCR Master Mix and ABI Prism 7000 Sequence Detection System (Applied Biosystems). After amplification, the specificity of the PCR products was verified by a melting curve analysis. Relative quantification of the target genes in comparison with the reference (RPLP0) was calculated using the following equation (Pfaffl, M. W. “A New Mathematical Model for Relative Quantification in Real-time RT-PCR,” Nucleic Acids Res., 29: E45-E45 (2001)):
Ratio=(E_{target})^{ΔCP target (control-sample)}/(E_{ref})^{ΔCP ref (control-sample)}

Human prostate cancer cell line LNCaP was used as a positive control for RAR and RXR expressions (Blutt, S. E., Allegretto, E. A., Pike, J. W., and Weigel, N. L., “1,25-dihydroxyvitamin D3 and 9-cis-retinoic Acid Act Synergistically to Inhibit the Growth of LNCaP Prostate Cells and Cause Accumulation of Cells in G1,” Endocrinology, 138: 1491-1497 (1997)).

Expression of VDR

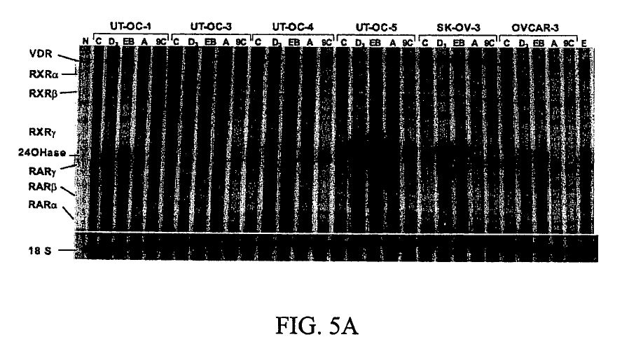

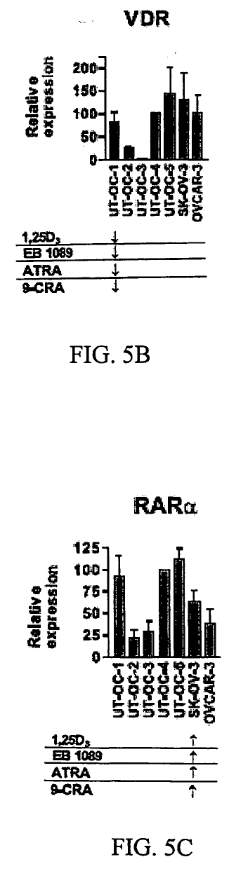

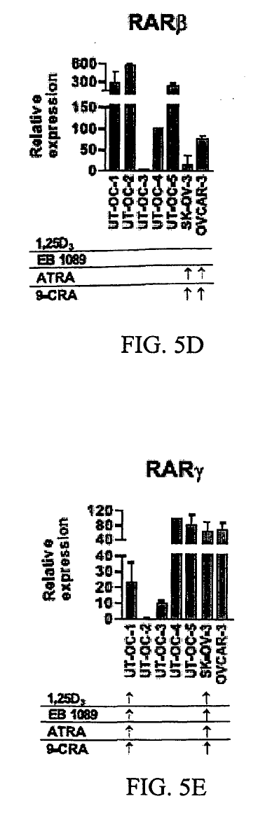

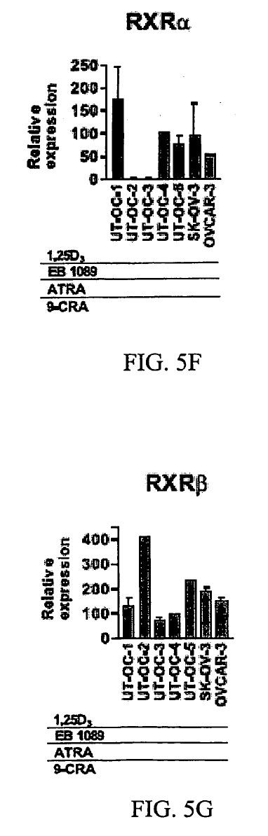

To study how receptor expression is connected with the growth responses, we analysed the basal expression level of VDR mRNA using RPA and quantitative RT-PCR methods. The effect of 1,25(OH)₂D₃, EB 1089, ATRA and 9-CRA on the expression of VDR mRNA was also tested. FIG. 5A is a scan of a gel electrophoresis shows the RPA results on VDR expression in ovarian cancer cell lines.

All cell lines studied expressed VDR mRNA (FIG. 5B). The most abundant expression was in UT-OC-5 cells, the least abundant in cell line UT-OC-3. The VDR expression did not correlate with the growth responses to vitamin D compounds. The expression of VDR mRNA was similar in cell lines UT-OC-1, UT-OC4, UT-OC-5, SK-OV-3 and OVCAR-3, but they differed in their growth responses to 1,25(OH)₂D₃ and EB 1089. 1,25(OH)₂D₃, EB 1089, ATRA and 9-CRA down regulated VDR in UT-OC-1 cells (36±17, 47±8, 53±11 and 58±25% of control, respectively). In other cell lines VDR expression was not regulated.

Expression of RARs and RXRs

Quantitative RT-PCR and RPA methods were also used to analyse the basal expression levels of RAR (α, β and γ) and RXR (α, β and γ) mRNA. We also tested whether the receptors were regulated by 1,25(OH)₂D₃, EB 1089, ATRA and 9-CRA. FIG. 5A show the RPA results on the expressions of these receptors in ovarian cancer cell lines. Spearman's non-parametric rank correlation test was used to detect correlations between RAR/RXR/VDR expressions and coregulator expressions or growth responses to ATRA or 9-CRA (Table III).

TABLE III
Correlations of receptor and cofactor expressions
in ovarian cancer cell lines.

| | |
|---|---|
| Receptor | Correlation^{a} |

| | | | |
|---|---|---|---|
| RARα | RXRα | AIB1 | NCoR |
| | r = 0.78 | r = 0.70 | r = −0.74 |
| | (0.41-0.93) | (0.25-0.90) | (−0.92-(−0.34)) |
| | P = 0.001 | P = 0.0057 | P = 0.0023 |
| RARγ | VDR | ATRA/Growth | 9-CRA/Growth |
| | r = 0.64 | r = 0.58 | r = 0.54 |
| | (0.15-0.88) | (0.05-0.85) | (0.002-0.84) |
| | P = 0.013 | P = 0.03 | P = 0.046 |
| RXRα | RARα | AIB1 | |
| | r = 0.78 | r = 0.81 | |
| | (0.41-0.93) | (0.47-0.94) | |
| | P = 0.001 | P = 0.0005 | |
| RXRβ | TIF2 | | |
| | r = 0.69 | | |
| | (0.23-0.90) | | |
| | P = 0.0066 | | |

^{a}Spearman's rank correlation test
95% confidence intervals are indicated within parenthesis

RARα expression was strongest in UT-OC-5 cells (FIG. 5C). The lowest expression was in UT-OC-2 cells. In SK-OV-3 cells, RARα was up-regulated by 1,25(OH)₂D₃, EB 1089, ATRA and 9-CRA (186±8, 134±10, 293±70 and 195±1% of control, respectively). The amount of RARβ varied considerably between cell lines (FIG. 5D). The expression was strongest in UT-OC-2 cells and weakest in UT-OC-3 cells. ATRA and 9-CRA increased the expression of RARβ in SK-OV-3 cells (657±30 and 345±17% of control) and in OVCAR-3 cells (214±127 and 276±68% of control).

The RARγ expression was strongest in UT-OC-4 and weakest in UT-OC-2 cells (FIG. 5E). The amount of RARγ was up-regulated in SK-OV-3 and UT-OC-1 cells by vitamin D and retinoids. In SK-OV-3 cells 100 nM 1,25(OH)₂D₃ increased RARγ by 78±12%, 100 nM EB 1089 by 51±7%, 10 μM ATRA by 119±19% and 10 μM 9-CRA also by 119±52%, when compared to control. In UT-OC-1 cells 100 nM 1,25(OH)₂D₃ up regulated RARγ by 91±81%, 100 nM EB 1089 by 47±7%, 10 μM ATRA by 83±66% and 10 μM 9-CRA by 44±15%, when compared to control. RXRα expression was most marked in UT-OC-1 cells and lowest in UT-OC-2 and UT-OC-3 cells (FIG. 5F). There was extensive variation between cell lines in RXRβ expression (FIG. 5G). It was expressed most abundantly in UT-OC-2 cells while in UT-OC-3 cells the expression was the lowest. We detected low levels of RXRγ in cell lines UT-OC-1, UT-OC-2, UT-OC-5 and OVCAR-3 (data not shown). In other lines the receptor was either not expressed or the expression was under the detection limit of the quantitative real-time PCR method.

Expression of Coregulators

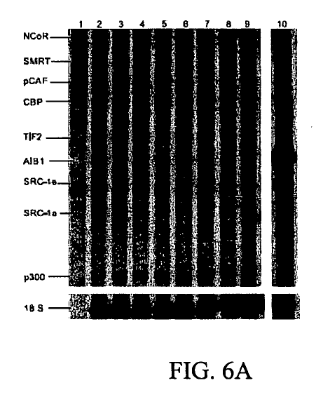

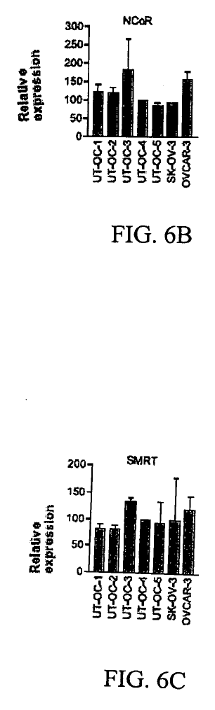

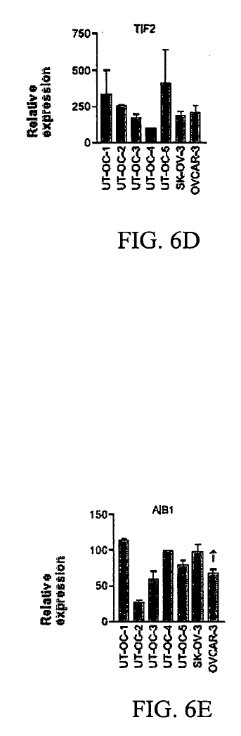

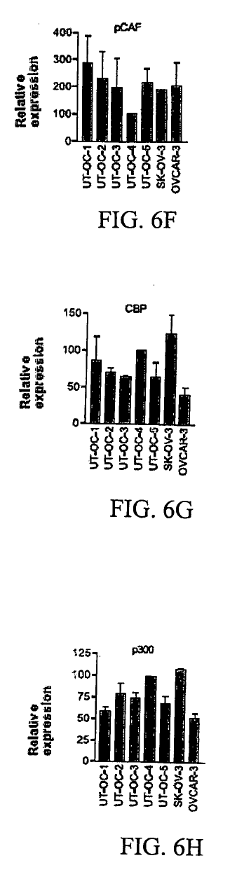

The differential expression pattern of nuclear receptor cofactors in cells might also affect cellular responses to hormones. FIG. 6A is a scan of a gel electrophorsesis showing the RPA results on nuclear receptor coregulator expressions in ovarian cancer cell lines.

The basic coregulator expression pattern was similar in all seven ovarian cancer cell lines. FIGS. 6B-H show the expression of different cofactors in cell lines. The nuclear receptor coinhibitors NCoR and SMRT were most abundantly expressed in the UT-OC-3 cell line. The cointegrator pCAF was most abundant in UT-OC-1 cells and CBP and p300 in SK-OV-3 cells. The expression levels of CBP and p300 in cells correlated (r=0.66, 95% CI 0.18-0.88, P=0.011). The expression of coactivator TIF2 was strongest in UT-OC-5 cells. The expression of AIB1 was strongest in UT-OC-1 cells. In OVCAR-3 cells ATRA and 9-CRA slightly up-regulated AIB1 expression (160 (±10) and 171 (±5) % of ethanol-treated control, respectively). As the basal expression of SRC-1 in cells was low, it could not be quantified. Both SRC-1 isoforms, -1a and -1e, are expressed in the ovarian cancer cell lines studied.

Expression of 24OHase

We studied the expression and induction of 24OHase by vitamin D₃ and retinoid compounds in ovarian cancer cells. The RPA results on the expression and induction of 24OHase on cell lines are shown in FIG. 5A.

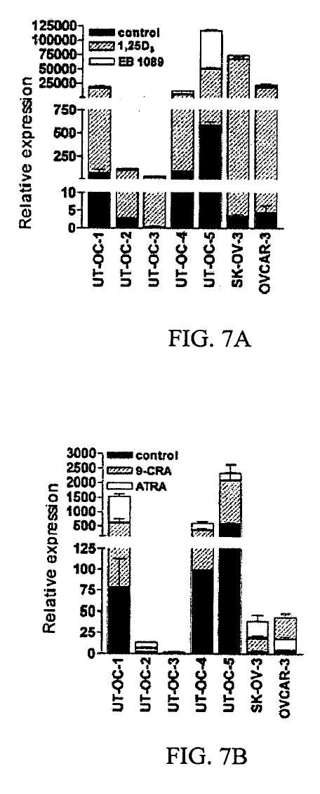

All cell lines expressed the 24OHase enzyme, but the basic expression levels varied (FIGS. 7A and B). This enzyme was most abundantly expressed in UT-OC-5 cells, in which the expression was six times stronger than in UT-OC-4 cells. The expression was lowest in UT-OC-3 cells (0.4% of UT-OC-4 expression). Table IV shows the results of a Spearman's rank correlation analysis. The basal expression levels of 24OHase correlated with receptors RARα and RXRα and co-activator AIB1. There was a negative correlation with co-inhibitor NCoR. There was no correlation between growth responses to calcitriol or retinoids and basal expression levels of 24OHase.

TABLE IV
Correlation^{a} of basal and induced 24OHase expression levels with receptor and cofactor expressions.

| | | 24OHase | | | | | |
|---|---|---|---|---|---|---|---|
| | | (Basal) | | RARα | RXRα | AIB1 | NCoR |
| Basal | | | | *** | ** | * | * |
| | 24OHase | | | r = 0.93, | r = 0.71, | r = 0.65, | r = −0.65, |
| | | | | P < 0.0001, | P = 0.0046, | P = 0.0115, | P = 0.0115, |
| | | | | (0.79-0.98) | (0.27-0.90) | (0.17-0.88) | (−0.88-−0.17) |
| Induced | 1,25D₃ | * | *** | * | — | * | ** |
| | (100 nM) | r = 0.57, | r = 0.89, | r = 0.60, | | r = 0.54, | r = −0.66, |
| | | P = 0.035, | P < 0.0001, | P = 0.03, | | P = 0.0475, | P = 0.0098, |
| | | (0.03-0.85) | (0.67-0.97) | (0.05-0.85) | | (0.01-0.84) | (−0.89-−0.19) |
| | EB 1089 | ** | *** | ** | — | — | * |
| | (100 nM) | r = 0.68, | r = 0.86, | r = 0.68, | | | r = −0.65, |
| | | P = 0.0073, | P < 0.0001, | P = 0.0078, | | | P = 0.0126, |
| | | (0.23-0.89) | (0.60-0.96) | (0.21-0.89) | | | (−0.88-−0.16) |
| | ATRA | *** | — | *** | ** | ** | ** |
| | (10 μM) | r = 0.95, | | r = 0.95, | r = 0.74, | r = 0.72, | r = −0.75, |
| | | P < 0.0001, | | P < 0.0001, | P = 0.0023, | P = 0.0039, | P = 0.0021, |
| | | (0.83-0.98) | | (0.84-0.99) | (0.34-0.92) | (0.29-0.91) | (−0.92-−0.35) |
| | 9-CRA | *** | — | *** | ** | * | * |
| | (10 μM) | r = 0.93, | | r = 0.92, | r = 0.74, | r = 0.60, | r = −0.59, |
| | | P < 0.0001, | | P < 0.0001, | P = 0.0025, | P = 0.0238, | P = 0.0265, |
| | | (0.78-0.98) | | (0.76-0.98) | (0.33-0.92) | (0.08-0.86) | (−0.86-−0.07) |

^{a}Spearman's rank correlation test
95% confidence intervals are indicated within parenthesis
— No correlation,
* weak,
** moderate,
*** strong correlation

Induction of 24OHase Expression

Also the induction levels of 24OHase varied extensively. On every cell line the vitamin D₃ analogue EB 1089 proved to be more effective in inducing 24OHase expression (FIG. 7A). This was most prominent in UT-OC-5 cells, where 100 nM EB 1089 was a more than two-fold stronger inducer than 100 nM 1,25(OH)₂D₃. Although the basal expression of the enzyme was low in SK-OV-3 cells, the induction was most prominent. Likewise in OVCAR-3 we could detect high induction levels of the enzyme although the basal expression was relatively low. Induction of 24OHase mRNA by vitamin D₃ compounds correlated strongly with the VDR expressions of cell lines (Table IV). The basal expression of 24OHase correlated weakly with 1,25(OH)₂D₃ induction levels and moderately with EB 1089 induction levels of 24OHase. The inductions of 24OHase by 1,25(OH)₂D₃ and EB 1089 were strongly intercorrelated (r=0.93, 95% CI 0.78-0.98, P<0.0001). The induction levels of 24OHase by 1,25(OH)₂D₃ correlated with AIB1 expression levels, and those of 24OHase by 1,25(OH)₂D₃ and EB 1089 also correlated negatively with NCoR expression in cells (Table IV).

Expression of 24OHase was also induced by retinoids, although to a lesser degree than with 1,25(OH)₂D₃ and EB 1089 (FIG. 7B). In all cell lines except OVCAR-3 the all-trans isomer induced the expression of 24OHase more than 9-cis retinoic acid. This was most obvious in UT-OC-1 cells, where 10 μM ATRA was a more than three-fold stronger inducer than 10 μM 9-CRA. The basal expression of 24OHase correlated markedly with ATRA and 9-CRA induction levels of 24OHase (Table IV). The induction of 24OHase by ATRA and 9-CRA correlated strongly with RARα and moderately with RXRα. The induction of 24OHase by ATRA and 9-CRA were strongly intercorrelated (r=0.95, 95% CI 0.85-0.99, P<0.0001). The induction levels of 24OHase by ATRA and 9-CRA correlated positively with AIB1 and negatively with NCoR expression levels (Table IV). There was no correlation between the growth responses to calcitriol or retinoids and the induced expression levels of 24OHase.

Effect of VID400 on Growth Response

The effect of the inhibition of the enzymatic activity of 24OHase on growth response to 1,25(OH)₂D₃, EB 1089 and retinoids. We studied combination of 100 nM 1,25(OH)₂D₃, 100 nM EB 1089, 10 μM ATRA or 10 μM 9-CRA with a specific 24OHase inhibitor, VID 400 (200 nM). In cell lines SK-OV-3 and OVCAR-3 we also tested a combination of 10 nM 1,25(OH)₂D₃, 10 nM EB 1089, 1 μM ATRA or 1 μM 9-CRA with 200 nM VID 400.

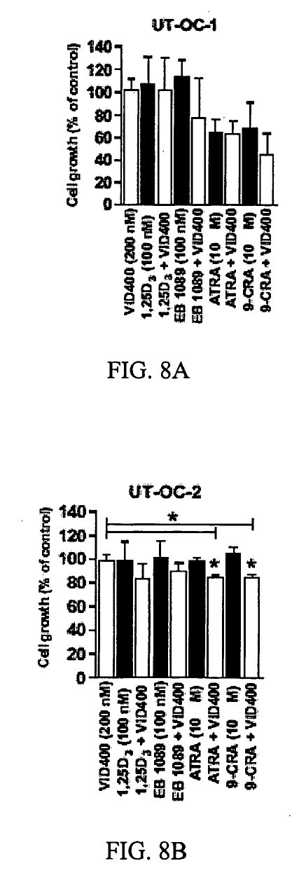

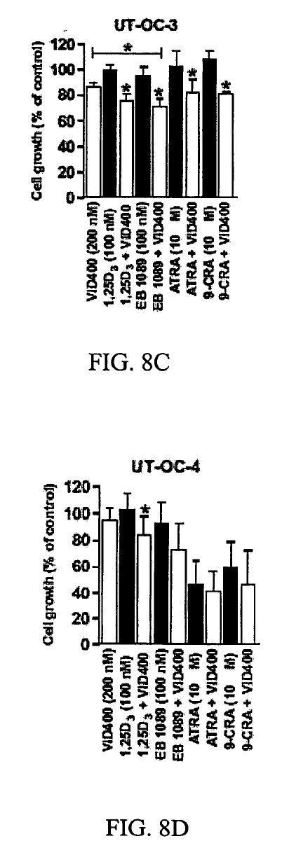

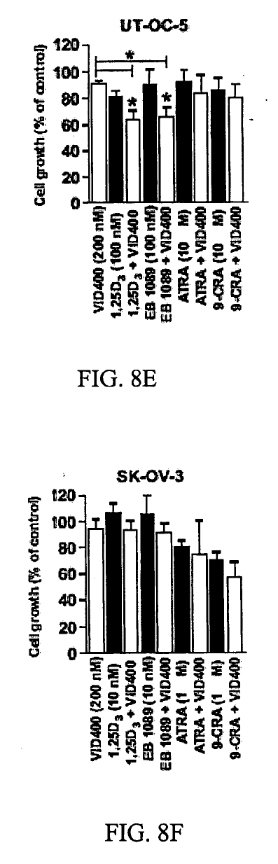

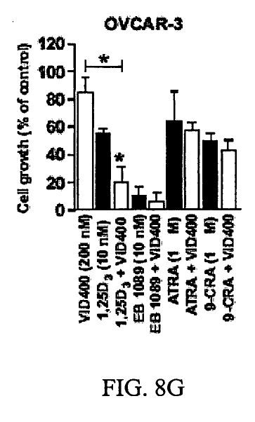

The effect of VID 400 was cell line-specific (FIGS. 8A-G). In cell line UT-OC-1, 1,25(OH)₂D₃ or EB 1089 alone did not inhibit the cell growth, but the combination of VID 400 and EB 1089 did so. Both ATRA and 9-CRA alone inhibited cell growth with equal magnitude. When VID 400 was combined with 9-CRA, it augmented the growth inhibition, but this was not seen with ATRA. In UT-OC-2 and UT-OC-3 cells neither vitamin D nor retinoid compounds alone had an effect on cell growth; however when combined with VID 400 growth inhibition was seen. Also in UT-OC-4 cells 1,25(OH)₂D₃ or EB 1089 alone did not restrain cell growth, but in combination with VID 400 cell growth was inhibited. Both ATRA and 9-CRA inhibited UT-OC-4 cell growth and the combination with VID 400 potentiated this effect. The growth of UT-OC-5 cells was not clearly inhibited by any of the compounds tested, but combination of hormones, especially 1,25(OH)₂D₃ or EB 1089, with VID 400 had an inhibitory effect on growth. In addition to the above, the combinations, 10 mM 1,25(OH)₂D₃, 10 nM EB 1089, 1 μM ATRA or 1 μM 9-CRA with 200 nM VID 400 in SK-OV-3 and OVCAR-3 cells were tested. The results with both concentrations were similar. In SK-OV-3 cells vitamin D compounds did not inhibit cell growth, but in combination with VID 400 they exerted a slightly inhibitory effect on growth. Both ATRA and 9-CRA inhibited the growth of SK-OV-3 cells and the combination of 9-CRA with VID 400 potentiated this effect. In OVCAR-3 cells VID 400 clearly enhanced the growth inhibition induced by 10 nM 1,25(OH)₂D₃. EB 1089 alone markedly inhibited the proliferation of OVCAR-3 cells and consequently the combination of EB 1089 with VID 400 had no additional effect on cell growth inhibition. When used in combination with retinoids VID 400 slightly augmented the growth inhibition in OVCAR-3 cells. As shown in FIGS. 8A-G, the effect of VID 400 alone in the cell lines varied from slight stimulation to growth inhibition.

Experiment VI

Inhibition of Cancer Cell Growth by VID400-1,25(OH)₂D₃ Cotherapy.

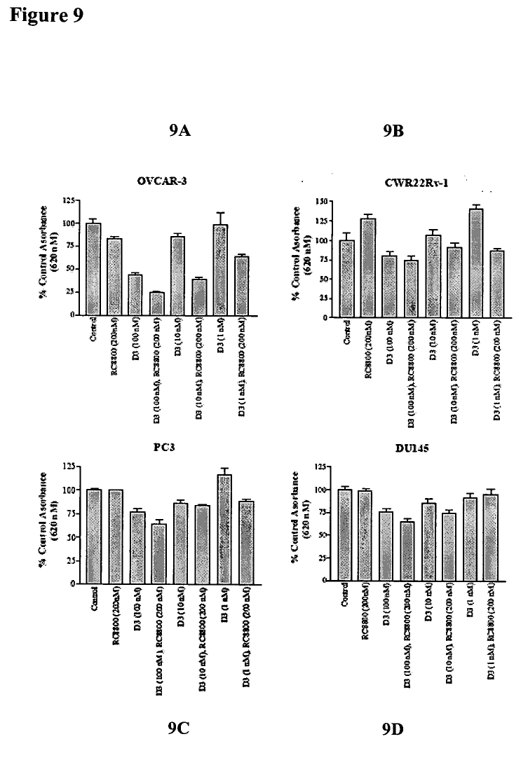

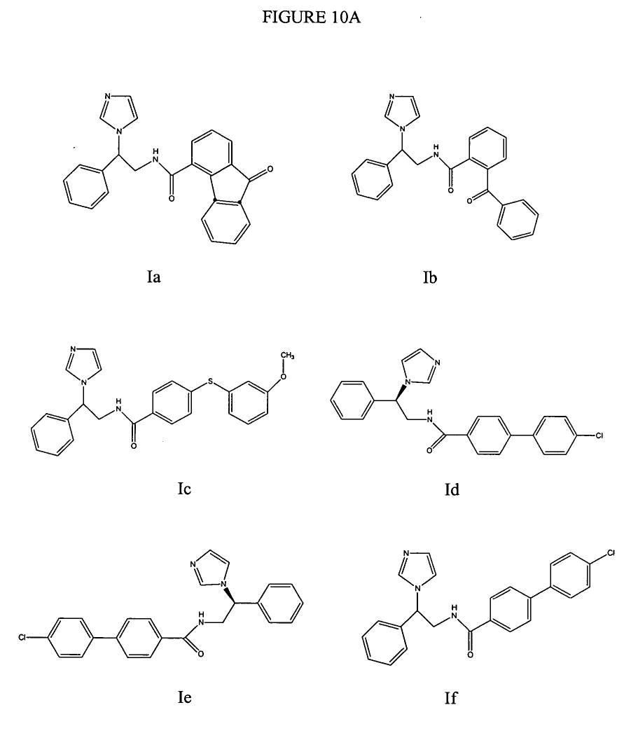

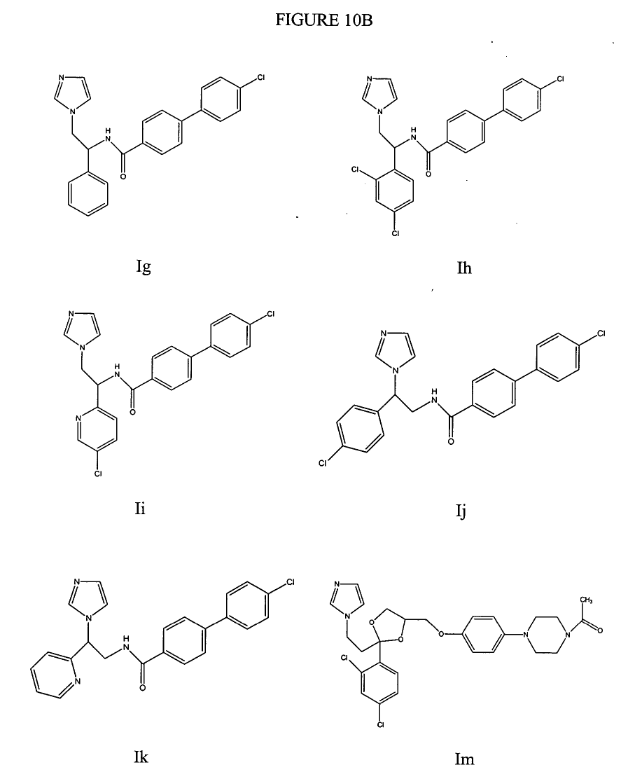

Inhibition of the growth of one ovarian cancer cell line and three different prostate cancer cell lines by VID400 (also known as RC-8800), 1,25(OH)₂D₃, and VID400-1,25(OH)₂D3 cotherapy was investigated using methods similar to those described in Experiment I. The results are depicted in FIGS. 9A-9D. Culture conditions were the same as for Experiment I. Cell growth was measured using uptake of methylene blue (0.5% in 50% ethanol), followed by air drying and release of the dye in 1% sodium N-lauroyl sarcosine in PBS.

FIG. 9A shows similar results for the ovarian cancer cell line OVCAR-3 as obtained in Experiment I. Calcitriol inhibited cell growth in a concentration-dependent manner, and 200 nM VID400 produced a further inhibition which was synergistic with calcitriol. Prostate cancer cell lines CWR22Rv-1 (FIG. 9B), PC3 (FIG. 9C), and DU145 (FIG. 9D) each showed a similar pattern of synergistic inhibition of growth by VID400+calcitriol, although the effect of calcitriol alone was less pronounced due to the resistance of these cells to calcitriol alone.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating cancer in a subject in need thereof comprising administering a therapeutically effective amount of a 24-hydroxylase inhibitor, wherein the 24-hydroxylase inhibitor is represented by the structural Formula I: 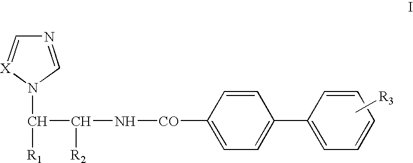
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄) alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

2. The method of claim 1, wherein the 24-hydroxylase inhibitor is represented by the structural Formula II: 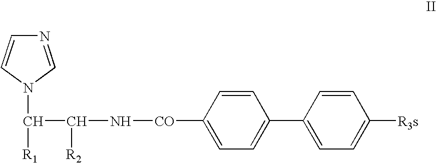
or a pharmaceutically acceptable salt, solvate or hydrate thereof, wherein:
R₁ₛ is phenyl, phenyl monosubstituted by halogen, or 1-naphtyl, and R₂ₛ is hydrogen; or
R₁ s is hydrogen and R₂ₛ is pyridyl or 2-(5-chloro)pyridyl; and
R₃ₛ is halogen, (C₁₋₄) alkoxy.

3. The method of claim 1, wherein the 24-hydroxylase inhibitor is selected from the group consisting of structures 1d and 1e of FIG. 10, or a pharmaceutically acceptable salt, solvate or hydrate thereof.

4. The method of claim 1, wherein said inhibitor is a compound selected from the group consisting of azoles, aminoalkanimidazoles, aminoalkantriazoles, acylated aminoalkanimidazoles, and acylated aminoalkantriazoles.

5. The method of claim 1, wherein said inhibitor is selected from the group consisting of ketoconazole, clotrimazole, fluconazole, itraconazole, and liarozole.

6. The method of claim 1, wherein said azole compound has a bulky substituent attached at the carbon atom which is in the alpha position relative to the azole.

7. The method of claim 6, wherein said substituent is phenyl, naphthyl, thienyl, or pyridyl; or phenyl, naphthyl, thienyl or pyridyl monosubstituted by halogen, (C1-4) alkoxy, (C1-4)alkyl, di-(C1-4)alkylamino or cyano.

8. The method of claim 7, wherein said inhibitor is selected from the group consisting of (R)-SDZ-286907, (R)-SDZ-287871, (R)-VAB636, (R)-VID400, and (S)-SDZ-285428.

9. The method of claim 1, wherein said inhibitor is represented by Formula IV 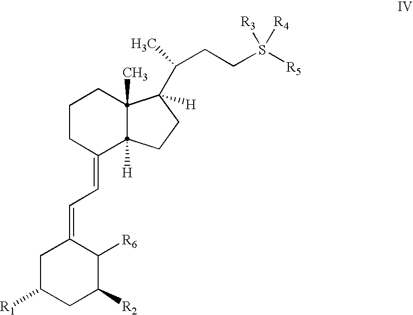
wherein R1 and R2 are each independently selected from the group consisting of hydrogen, OR′, —C(O)H, and —C(O)R′;
wherein R′ is selected from the group consisting of a C1 to C6 alkyl, a cycloalkyl, phenyl, an alkylaryl, an arylalkyl, and a heteroaryl; each of which can be optionally substituted with at least one halogen, thiol, mercapto, hydroxyl, or amino group;
wherein R3, R4 and R5 are each independently selected from the group consisting of hydrogen, hydroxyl, oxy, imine, phenyl, a C1 to C6 alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R6 is hydrogen, ═CH2 or a C1 to C6 alkyl, alkenyl, cycloalkyl, or cycloalkenyl, each of which can be optionally substituted with at least one halogen, thiol, mercapto, hydroxyl, or amino group;
or a pharmaceutically acceptable salt, hydrate, solvate, ester, or isomer thereof.

10. The method of claim 9, wherein said inhibitor is represented by Formula V 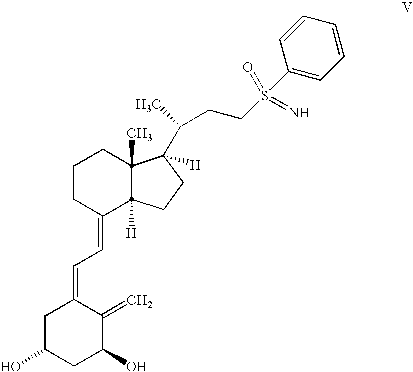
or a pharmaceutically acceptable salt, solvate, hydrate, ester or isomer thereof.

11. The method of claim 9, wherein said inhibitor is represented by Formula VI 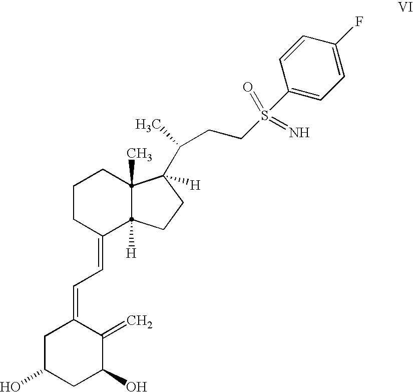
or a pharmaceutically acceptable salt, solvate, hydrate, ester or isomer thereof.

12. The method of claim 9, wherein said inhibitor is selected from the group consisting of compounds IIa, IIb, IIc, IId, IIe, IIf, IIg, IIh, IIi, IIj, and IIk as shown in FIG. 11 and pharmaceutically acceptable salts, solvates, hydrates, esters, or isomers thereof.

13. The method of claim 1, wherein the 24-hydroxylase inhibitor is administered orally.

14. A method for treating cancer in a subject in need thereof comprising administering to said subject:
i) a first amount of a 24-hydroxylase inhibitor; and
ii) a second amount of calcitriol
wherein the first and second amounts together comprise a therapeutically effective amount.

15. The method of claim 14, wherein the 24-hydroxylase inhibitor is represented by the structural Formula I: 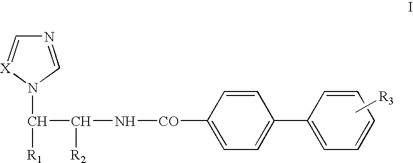
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄) alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

16. The method of claim 14, wherein the 24-hydroxylase inhibitor is represented by the structural Formula II: 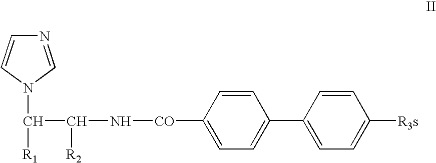
or a pharmaceutically acceptable salt, solvate or hydrate thereof, wherein:
R₁ₛ is phenyl, phenyl monosubstituted by halogen, or 1-naphtyl, and R₂ₛ is hydrogen; or
R₁ₛ is hydrogen and R₂ₛ is pyridyl or 2-(5-chloro)pyridyl; and
R₃ₛ is halogen, (C₁₋₄) alkoxy.

17. The method of claim 14, wherein the 24-hydroxylase inhibitor is selected from the group consisting of structures 1d and 1e of FIG. 10, or a pharmaceutically acceptable salt, solvate or hydrate thereof.

18. The method of claim 14, wherein said inhibitor is a compound selected from the group consisting of azoles, aminoalkanimidazoles, aminoalkantriazoles, acylated aminoalkanimidazoles, and acylated aminoalkantriazoles.

19. The method of claim 14, wherein said inhibitor is selected from the group consisting of ketoconazole, clotrimazole, fluconazole, itraconazole, and liarozole.

20. The method of claim 14, wherein said azole compound has a bulky substituent attached at the carbon atom which is in the alpha position relative to the azole.

21. The method of claim 20, wherein said substituent is phenyl, naphthyl, thienyl, or pyridyl; or phenyl, naphthyl, thienyl or pyridyl monosubstituted by halogen, (C1-4) alkoxy, (C1-4)alkyl, di-(C1-4)alkylamino or cyano.

22. The method of claim 21, wherein said inhibitor is selected from the group consisting of (R)-SDZ-286907, (R)-SDZ-287871, (R)-VAB636, (R)-VID400, and (S)-SDZ-285428.

23. The method of claim 14, wherein said inhibitor is represented by Formula IV 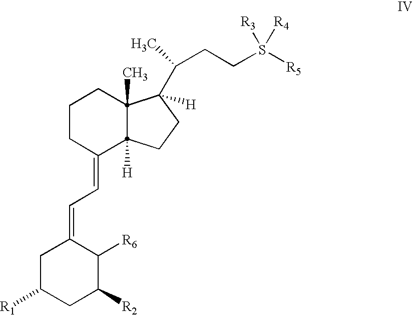
wherein R1 and R2 are each independently selected from the group consisting of hydrogen, OR′, —C(O)H, and —C(O)R′;
wherein R′ is selected from the group consisting of a C1 to C6 alkyl, a cycloalkyl, phenyl, an alkylaryl, an arylalkyl, and a heteroaryl; each of which can be optionally substituted with at least one halogen, thiol, mercapto, hydroxyl, or amino group;
wherein R3, R4 and R5 are each independently selected from the group consisting of hydrogen, hydroxyl, oxy, imine, phenyl, a C1 to C6 alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R6 is hydrogen, ═CH2 or a C1 to C6 alkyl, alkenyl, cycloalkyl, or cycloalkenyl, each of which can be optionally substituted with at least one halogen, thiol, mercapto, hydroxyl, or amino group;
or a pharmaceutically acceptable salt, hydrate, solvate, ester, or isomer thereof.

24. The method of claim 23, wherein said inhibitor is represented by Formula V 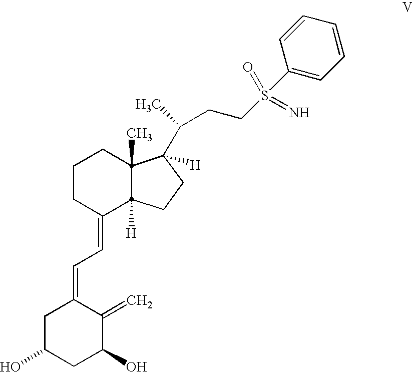
or a pharmaceutically acceptable salt, solvate, hydrate, ester or isomer thereof.

25. The method of claim 23, wherein said inhibitor is represented by Formula VI 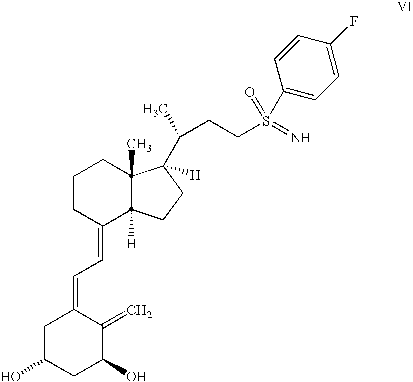
or a pharmaceutically acceptable salt, solvate, hydrate, ester or isomer thereof.

26. The method of claim 23, wherein said inhibitor is selected from the group consisting of compounds IIa, IIb, IIc, IId, IIe, IIf, IIg, IIh, IIi, IIj, and IIk as shown in FIG. 11 and pharmaceutically acceptable salts, solvates, hydrates, esters, or isomers thereof.

27. The method of claim 14, wherein the 24-hydroxylase inhibitor is administered orally.

28. A method of treating cancer selected from the group consisting of colorectal cancer, esophageal cancer, myelodysplastic syndrome, multiple myeloma, gliomas, non-small cell lung cancer, stomach cancer, acute myeloid leukemia, hepatocellular carcinoma, breast cancer, ovarian cancer or prostate cancer in a subject in need thereof comprising administering a therapeutically effective amount of a 24-hydroxylase inhibitor, wherein the 24-hydroxylase inhibitor is represented by the structural Formula I: 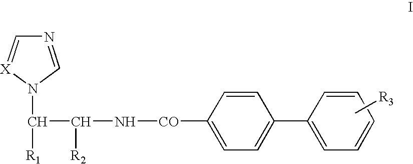
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄) alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

29. The method of claim 28, wherein the 24-hydroxylase inhibitor is represented by the structural Formula II: 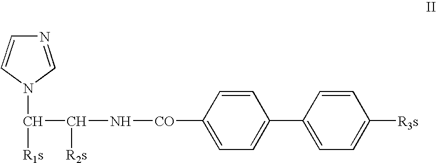
or a pharmaceutically acceptable salt, solvate or hydrate thereof, wherein:
R₁ₛ is phenyl, phenyl monosubstituted by halogen, or 1-naphtyl, and R₂ₛ is hydrogen; or
R₁ₛ is hydrogen and R₂ₛ is pyridyl or 2-(5-chloro)pyridyl; and
R₃ₛ is halogen, (C₁₋₄) alkoxy.

30. The method of claim 28, wherein the 24-hydroxylase inhibitor is selected from the group consisting of structures 1d and 1e of FIG. 10, or a pharmaceutically acceptable salt, solvate or hydrate thereof.

31. The method of claim 28, wherein said inhibitor is a compound selected from the group consisting of azoles, aminoalkanimidazoles, aminoalkantriazoles, acylated aminoalkanimidazoles, and acylated aminoalkantriazoles.

32. The method of claim 28, wherein said inhibitor is selected from the group consisting of ketoconazole, clotrimazole, fluconazole, itraconazole, and liarozole.

33. The method of claim 28, wherein said azole compound has a bulky substituent attached at the carbon atom which is in the alpha position relative to the azole.

34. The method of claim 33, wherein said substituent is phenyl, naphthyl, thienyl, or pyridyl; or phenyl, naphthyl, thienyl or pyridyl mono substituted by halogen, (C₁-4) alkoxy, (C₁₋₄)alkyl, di-(C1-4)alkylamino or cyano.

35. The method of claim 34, wherein said inhibitor is selected from the group consisting of (R)-SDZ-286907, (R)-SDZ-287871, (R)-VAB636, (R)-VID400, and (S)-SDZ-285428.

36. The method of claim 28, wherein said inhibitor is represented by Formula IV 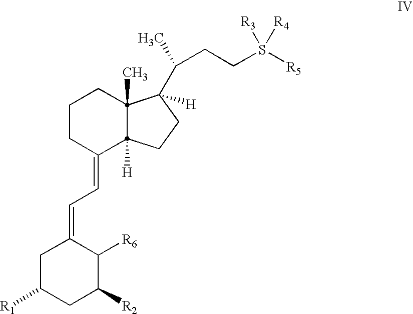
wherein R1 and R2 are each independently selected from the group consisting of hydrogen, OR′, —C(O)H, and —C(O)R′;
wherein R′ is selected from the group consisting of a C1 to C6 alkyl, a cycloalkyl, phenyl, an alkylaryl, an arylalkyl, and a heteroaryl; each of which can be optionally substituted with at least one halogen, thiol, mercapto, hydroxyl, or amino group;
wherein R3, R4 and R5 are each independently selected from the group consisting of hydrogen, hydroxyl, oxy, imine, phenyl, a C1 to C6 alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R6 is hydrogen, ═CH2 or a C1 to C6 alkyl, alkenyl, cycloalkyl, or cycloalkenyl, each of which can be optionally substituted with at least one halogen, thiol, mercapto, hydroxyl, or amino group;
or a pharmaceutically acceptable salt, hydrate, solvate, ester, or isomer thereof.

37. The method of claim 36, wherein said inhibitor is represented by Formula V 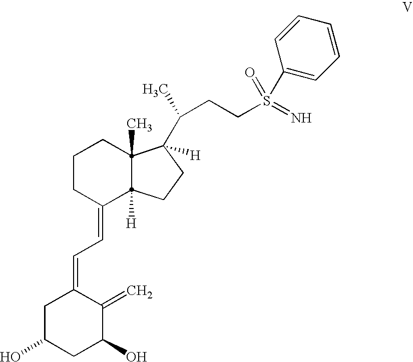
or a pharmaceutically acceptable salt, solvate, hydrate, ester or isomer thereof.

38. The method of claim 36, wherein said inhibitor is represented by Formula VI 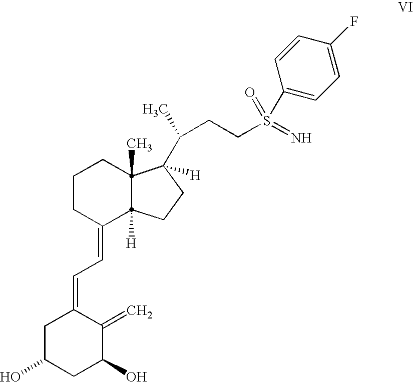
or a pharmaceutically acceptable salt, solvate, hydrate, ester or isomer thereof.

39. The method of claim 36, wherein said inhibitor is selected from the group consisting of compounds IIa, IIb, IIc, IId, IIe, IIf, IIg, IIh, IIi, IIj, and IIk as shown in FIG. 11 and pharmaceutically acceptable salts, solvates, hydrates, esters, or isomers thereof.

40. The method of claim 28, wherein the 24-hydroxylase inhibitor is administered orally.

41. A method of treating cancer selected from the group consisting of colorectal cancer, esophageal cancer, myelodysplastic syndrome, multiple myeloma, gliomas, non-small cell lung cancer, stomach cancer, acute myeloid leukemia, hepatocellular carcinoma, breast cancer, ovarian cancer or prostate cancer in a subject in need thereof comprising administering to said subject:
i) a first amount of a 24-hydroxylase inhibitor; and
ii) a second amount of calcitriol
wherein the first and second amount together comprise a therapeutically effective amount.

42. The method of claim 41, wherein the 24-hydroxylase inhibitor is represented by the structural Formula I: 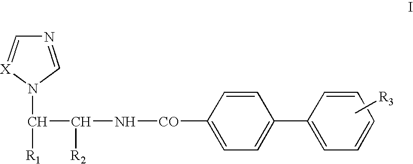
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄) alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

43. The method of claim 41, wherein the 24-hydroxylase inhibitor is represented by the structural Formula II: 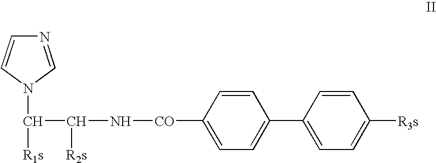
or a pharmaceutically acceptable salt, solvate or hydrate thereof, wherein:
R₁ₛ is phenyl, phenyl monosubstituted by halogen, or 1-naphtyl, and R₂ₛ is hydrogen; or
R₁ₛ is hydrogen and R₂ₛ is pyridyl or 2-(5-chloro)pyridyl; and
R₃ₛ is halogen, (C₁₋₄) alkoxy.

44. The method of claim 41, wherein the 24-hydroxylase inhibitor is selected from the group consisting of structures 1d and 1e of FIG. 10, or a pharmaceutically acceptable salt, solvate or hydrate thereof.

45. The method of claim 41, wherein said inhibitor is a compound selected from the group consisting of azoles, aminoalkanimidazoles, aminoalkantriazoles, acylated aminoalkanimidazoles, and acylated aminoalkantriazoles.

46. The method of claim 41, wherein said inhibitor is selected from the group consisting of ketoconazole, clotrimazole, fluconazole, itraconazole, and liarozole.

47. The method of claim 41, wherein said azole compound has a bulky substituent attached at the carbon atom which is in the alpha position relative to the azole.

48. The method of claim 47, wherein said substituent is phenyl, naphthyl, thienyl, or pyridyl; or phenyl, naphthyl, thienyl or pyridyl mono substituted by halogen, (C1-4) alkoxy, (C₁₋₄)alkyl, di-(C₁₋₄)alkylamino or cyano.

49. The method of claim 48, wherein said inhibitor is selected from the group consisting of (R)-SDZ-286907, (R)-SDZ-287871, (R)-VAB636, (R)-VID400, and (S)-SDZ-285428.

50. The method of claim 41, wherein said inhibitor is represented by Formula IV 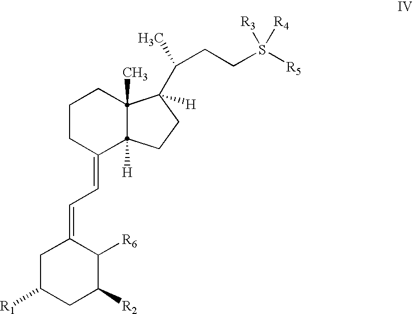
wherein R1 and R2 are each independently selected from the group consisting of hydrogen, OR′, —C(O)H, and —C(O)R′;
wherein R′ is selected from the group consisting of a C1 to C6 alkyl, a cycloalkyl, phenyl, an alkylaryl, an arylalkyl, and a heteroaryl; each of which can be optionally substituted with at least one halogen, thiol, mercapto, hydroxyl, or amino group;
wherein R3, R4 and R5 are each independently selected from the group consisting of hydrogen, hydroxyl, oxy, imine, phenyl, a C1 to C6 alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R6 is hydrogen, ═CH2 or a C1 to C6 alkyl, alkenyl, cycloalkyl, or cycloalkenyl, each of which can be optionally substituted with at least one halogen, thiol, mercapto, hydroxyl, or amino group;
or a pharmaceutically acceptable salt, hydrate, solvate, ester, or isomer thereof.

51. The method of claim 50, wherein said inhibitor is represented by Formula V 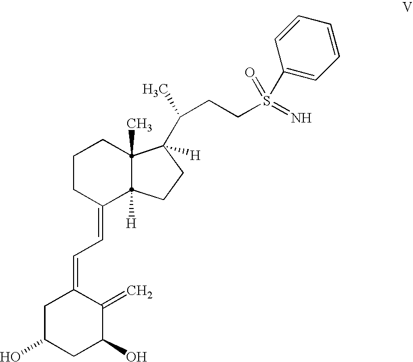
or a pharmaceutically acceptable salt, solvate, hydrate, ester or isomer thereof.

52. The method of claim 50, wherein said inhibitor is represented by Formula VI 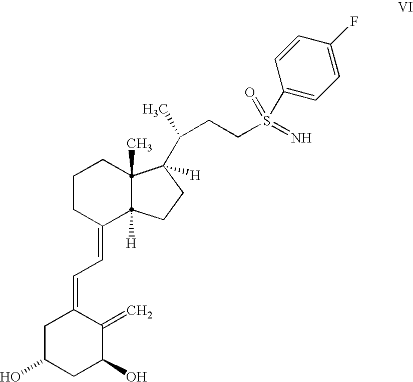
or a pharmaceutically acceptable salt, solvate, hydrate, ester or isomer thereof.

53. The method of claim 50, wherein said inhibitor is selected from the group consisting of compounds IIa, IIb, IIc, IId, IIe, IIf, IIg, IIh, IIi, IIj, and IIk as shown in FIG. 11 and pharmaceutically acceptable salts, solvates, hydrates, esters, or isomers thereof.

54. The method of claim 41, wherein the 24-hydroxylase inhibitor is administered orally.
